(19) United States
(12) Patent Application Publication
(10) Pub. No.: US 2019/0254973 A1
(43) Pub. Date: Aug. 22, 2019

(54) COMBINING ORTHOGONAL CHEMISTRIES FOR PREPARATION OF MULTIPLEXED NANOPARTICLES

(71) Applicant: Verily Life Sciences LLC, South San Francisco, CA (US)

(72) Inventors: Nicole Peck, Redwood City, CA (US); Zhan Wang, San Jose, CA (US)

(73) Assignee: Verily Life Sciences LLC, South San Francisco, CA (US)

(21) Appl. No.: 16/281,991

(22) Filed: Feb. 21, 2019

Related U.S. Application Data
(60) Provisional application No. 62/634,129, filed on Feb. 22, 2018.

(51) Int. Cl. A61K 9/127; C07K 16/32; C07K 16/28

(52) U.S. Cl. CPC A61K 9/1277; C07K 16/2863; C07K 16/32; A61K 9/1272

(57) ABSTRACT

Disclosed herein is a particle comprising a first effector element covalently bonded to a nanoparticle via a first orthogonal moiety and a second effector element covalently bonded to the nanoparticle via a second orthogonal moiety, wherein the first orthogonal moiety and the second orthogonal moiety have different chemical structures. The first effector element and the second effector element may be, for example, a targeting agent, a therapeutic agent, and a diagnostic agent. In certain embodiments, the nanoparticle is a liposome. Methods for preparing functionalized nanoparticles, including functionalized liposomes, are also described.

### CROSS-REFERENCE TO RELATED APPLICATION

The present application claims the benefit of priority to U.S. Provisional Application No. 62/634,129, filed on Feb. 22, 2018, the entire contents of which are incorporated by reference herein for all purposes.

### BACKGROUND OF THE INVENTION

Efficient delivery of nanoparticles to cells is important for their use in biomedical applications like drug delivery and imaging. Targeting nanoparticles to an overexpressed receptor on the cell has shown promise in cancer therapy, but most targeted nanomaterials are limited to a single receptor. This has a series of limitations related to morbidity and mortality of treatments due to nonspecific effects on normal cells. Many cancer cell types are distinct from healthy cells because they moderately overexpress multiple markers but do not overexpress a single receptor to 100-1000 fold above normal cells. These cells are difficult to target in a specific way using only one molecular recognition element.

Nanoparticles functionalized with two or more types of antibodies, peptides, or small molecules can provide a way to specifically target cells that overexpress multiple receptors. Simultaneous targeting could amplify the specificity a given nanoparticle has for a specific cell type. Particles with multiple molecular recognition elements (MREs) can provide control of complex signal pathways that require multiple target engagement. In addition to improved specificity for a particular cell type, multiple MREs on a single particle may allow for the binding and bringing together of two different cells (e.g, a killer T cell or a macrophage and a tumor cell). Furthermore, the ability to functionalize nanoparticles with two or more antibodies, peptides or small molecules can allow for hybrid particles with dual action, such as a targeting molecule for specificity and a therapeutic agent for treatment, or a therapeutic agent with a peptide or antibody that aids particle penetration through biological barriers. In order to achieve such outcomes, new nanoparticle compositions and methods for making them are needed. The present invention meets these needs.

### BRIEF SUMMARY OF THE INVENTION

Provided herein is a particle comprising a first effector element covalently bonded to a nanoparticle via a first orthogonal moiety and a second effector element covalently bonded to the nanoparticle via a second orthogonal moiety, wherein the first orthogonal moiety and the second orthogonal moiety have different chemical structures. The first effector element and the second effector element may be, for example, a targeting agent, a therapeutic agent, and a diagnostic agent. In some embodiments, the first orthogonal moiety and the second orthogonal moiety include a dihydropyridazine, a pyridazine, a triazole, a hydrazide, an oxime, a phosphoryl-substituted amide, or a thioether, provided that the second orthogonal moiety is different from the first orthogonal moiety.

Also provided here is a method of making a particle comprising a first effector element and a second effector element. The method includes:
(i) providing a nanoparticle having a first reactive functional group and a second reactive functional group; and
(ii) combining the nanoparticle with a first reactive effector element and a second reactive effector element under conditions sufficient to form:
   (a) a first orthogonal moiety covalent bonding the first reactive functional group to the first reactive recognition element and
   (b) a second orthogonal moiety covalently bonding the second reactive functional group to the second reactive effector element,
wherein the first orthogonal moiety and the second orthogonal moiety have different chemical structures; thereby forming the particle.

### BRIEF DESCRIPTION OF THE DRAWINGS

FIG. 1 shows a diagram of the general structure of a liposome with multiple orthogonal chemistries used to attach multiple targeting groups.

FIG. 2 shows a plot showing the incorporation of Tz-PEG-DSPE to produce liposomes with controlled amounts of desired surface functionality.

FIG. 3 shows a schematic of the synthesis of multiplexed liposomes by thin film hydration with PEG-lipids with different functional groups followed by conjugation of targeting groups.

FIG. 4 shows a schematic of the synthesis of multiplexed liposomes by post-insertion of PEG-lipid with different functional groups followed by conjugation of targeting groups.

FIG. 5 shows the binding of targeted liposomes to 1395, a cell line overexpressing EGFR.

FIG. 6 shows the binding of targeted liposomes to BT454, a cell line overexpressing HER2.

FIG. 7 shows the binding of targeted liposomes to ZR751, a cell line that has moderate expression of both EGFR and HER2.

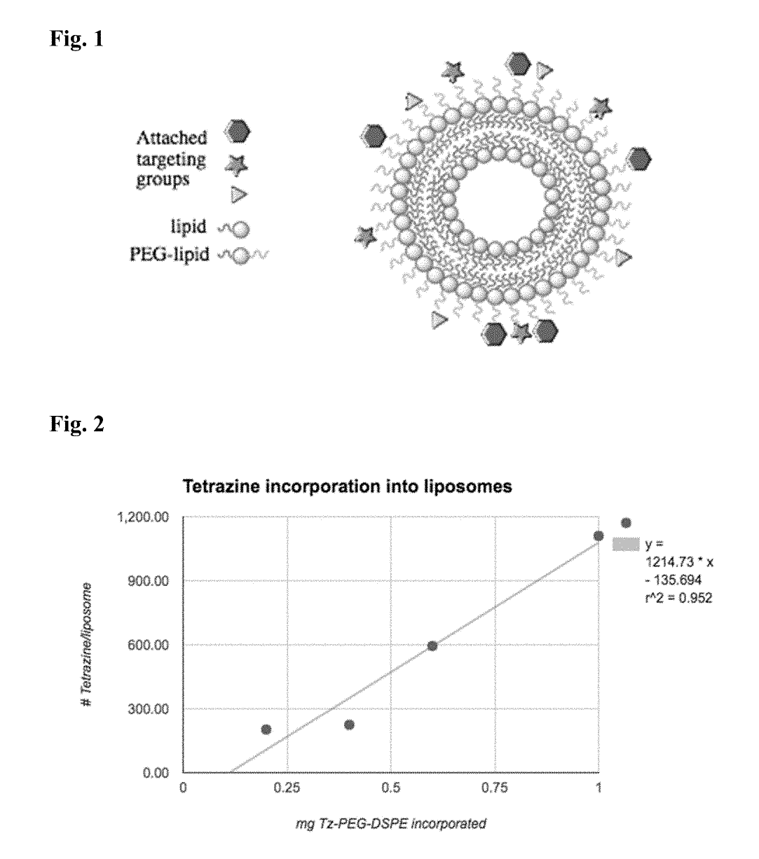

### DETAILED DESCRIPTION OF THE INVENTION

### I. GENERAL

Provided herein are nanoparticle compositions and methods which take advantage of multiple orthogonal chemistries to provide an exceptionally high degree of control over the conjugation of different functional elements (e.g., targeting groups for specific delivery of particle cargo to a target cells or tissues upon administration of the particles to a subject). As a non-limiting example, azide-modified antibodies will selectively react with alkyne-functionalized lipids, so the number of azide-modified antibodies that are presented on the surface of a liposome nanoparticle can be controlled by tuning the amount of the alkyne-functionalized lipid on the surface of the liposome.

There are several advantages to this method for producing multiplexed particles. This technology is not limited to combining two targeting groups. It can be expanded to include a variety of orthogonal chemistries, as described below, that can enable precise attachment of multiple molecules of interest. The attached molecules can also include very distinct targeting groups (e.g., full-length antibodies smaller molecules such as F_{ab} fragments) or a combination of targeting groups and diagnostic agents. Combining differently sized targeting groups is often a challenge as they are likely to conjugate at different rates. By using multiple orthogonal chemistries, competition is no longer a factor. This method also offers a targeting advantage, as targeting molecules can also target multiple types of cells while still taking advantage of avidity.

### II. DEFINITIONS

As used herein, the term “liposome” encompasses any compartment enclosed by a lipid bilayer. The term liposome includes unilamellar vesicles which are comprised of a single lipid bilayer and generally have a diameter in the range of about 20 nm to 10 μm. “Small unilamellar vesicles,” or SUVs typically range from about 20 nm to about 200 nm in size. Liposomes can also be multilamellar, which generally have a diameter in the range of 1 to 10

As used herein, the terms “liposome size” and “average particle size” refer to the outer diameter of a liposome. Average particle size can be determined by a number of techniques including dynamic light scattering (DLS), quasi-elastic light scattering (QELS), and electron microscopy.

As used herein, the term “polydispersity index” refers to the size distribution of a population of liposomes. Polydispersity index can be determined by a number of techniques including dynamic light scattering (DLS), quasi-elastic light scattering (QELS), and electron microscopy. Polydispersity index (PDI) is usually calculated as:

$$PDI = {( \frac{σ}{d} )}^{2}$$

i.e., the square of (standard deviation/mean diameter).

As used herein, the term “lipid” refers to lipid molecules that can include fats, waxes, steroids, cholesterol, fat-soluble vitamins, monoglycerides, diglycerides, phospholipids, sphingolipids, glycolipids, cationic or anionic lipids, derivatized lipids, and the like. Exemplary liposomes as described in detail below. Lipids can form micelles, monolayers, and bilayer membranes. The lipids can self-assemble into liposomes.

As used herein, the term “phosphatidylcholine” refers to a diacylglyceride phospholipid having a choline headgroup (i.e., a 1,2-diacyl-sn-glycero-3-phosphocholine). The acyl groups in a phosphatidylcholine lipid are generally derived from fatty acids having from 6 to 24 carbon atoms. Phosphatidylcholine lipids can include synthetic and naturally-derived 1,2-diacyl-sn-glycero-3-phosphocholines.

As used herein, the term “sterol” refers to a steroid containing at least one hydroxyl group. A steroid is characterized by the presence of a fused, tetracyclic gonane ring system. Sterols include, but are not limited to, cholesterol (i.e., 2,15-dimethyl-14-(1,5-dimethylhexyl)-tetracyclo[8.7.0.0^{2,7}.0^{11,15}]heptacos-7-en-5-ol; Chemical Abstracts Services Registry No. 57-88-5).

As used herein, the term “PEG-lipid” refers to a poly(ethylene glycol) polymer covalently bonded to a hydrophobic or amphiphilic lipid moiety. The lipid moiety can include fats, waxes, steroids, fat-soluble vitamins, monoglycerides, diglycerides, phospholipids, and sphingolipids. For example, the PEG-lipid may be a diacyl-phosphatidylethanolamine-N-[methoxy(polyethylene glycol)] or an N-acyl-sphingosine-1-{succinyl[methoxy(polyethylene glycol)]}. The molecular weight of the PEG in the PEG-lipid is generally from about 500 to about 5000 Daltons (Da; g/mol). The PEG in the PEG-lipid can have a linear or branched structure.

As used herein, the term “alkyl,” by itself or as part of another substituent, refers to a straight or branched, saturated, aliphatic radical having the number of carbon atoms indicated. Alkyl can include any number of carbons, such as C₁₋₂, C₁₋₃, C₁₋₄, C₁₋₅, C₁₋₆, C₁₋₇, C₁₋₈, C₁₋₉, C₁₋₁₀, C₂₋₃, C₂₋₄, C₂₋₅, C₂₋₆, C₃₋₄, C₃₋₅, C₃₋₆, C₄₋₅, C₄₋₆ and C₅₋₆. For example, C₁₋₆ alkyl includes, but is not limited to, methyl, ethyl, propyl, isopropyl, butyl, isobutyl, sec-butyl, tert-butyl, pentyl, isopentyl, hexyl, etc. Alkyl can also refer to alkyl groups having up to 20 carbons atoms, such as, but not limited to heptyl, octyl, nonyl, decyl, etc. Alkyl groups can be substituted or unsubstituted. Unless otherwise specified, “substituted alkyl” groups can be substituted with one or more groups selected from halo, hydroxy, amino, alkylamino, amido, acyl, nitro, cyano, and alkoxy.

As used herein, the term “alkylene” refers to an alkyl group, as defined above, linking at least two other groups (i.e., a divalent alkyl radical). The two moieties linked to the alkylene group can be linked to the same carbon atom or different carbon atoms of the alkylene group. The term “cycloalkylene” refers to a cycloalkyl group, as defined above, linking at least two other groups (i.e., a divalent cycloalkyl radical). The two moieties linked to the cycloalkylene group can be linked to the same carbon atom or different carbon atoms of the cycloalkylene group.

As used herein, the term “alkoxy,” by itself or as part of another substituent, refers to a group having the formula —OR, wherein R is alkyl.

As used herein the term “cycloalkyl,” by itself or as part of another substituent, refers to a saturated or partially unsaturated, monocyclic, fused bicyclic or bridged polycyclic ring assembly containing from 3 to 12 ring atoms, or the number of atoms indicated. Cycloalkyl can include any number of carbons, such as C₃₋₆, C₄₋₆, C₅₋₆, C₃₋₈, C₄₋₈, C₅₋₈, C₆₋₈, C₃₋₉, C₃₋₁₀, C₃₋₁₁, and C₃₋₁₂. Saturated monocyclic cycloalkyl rings include, for example, cyclopropyl, cyclobutyl, cyclopentyl, cyclohexyl, and cyclooctyl. Saturated bicyclic and polycyclic cycloalkyl rings include, for example, norbornane, [2.2.2] bicyclooctane, decahydronaphthalene and adamantane. Cycloalkyl groups can also be partially unsaturated, having one or more double or triple bonds in the ring. Representative cycloalkyl groups that are partially unsaturated include, but are not limited to, cyclobutene, cyclopentene, cyclohexene, cyclohexadiene (1,3- and 1,4-isomers), cycloheptene, cycloheptadiene, cyclooctene, cyclooctadiene (1,3-, 1,4- and 1,5-isomers), norbornene, and norbornadiene. When cycloalkyl is a saturated monocyclic C₃₋₈ cycloalkyl, exemplary groups include, but are not limited to cyclopropyl, cyclobutyl, cyclopentyl, cyclohexyl, cycloheptyl and cyclooctyl. When cycloalkyl is a saturated monocyclic C₃₋₆ cycloalkyl, exemplary groups include, but are not limited to cyclopropyl, cyclobutyl, cyclopentyl, and cyclohexyl. Cycloalkyl groups can be substituted or unsubstituted. Unless otherwise specified, “substituted cycloalkyl” groups can be substituted with one or more groups selected from halo, hydroxy, amino, alkylamino, amido, acyl, nitro, cyano, and alkoxy. The term “lower cycloalkyl” refers to a cycloalkyl radical having from three to seven carbons including, for example, cyclopropyl, cyclobutyl, cyclopentyl, cyclohexyl, and cycloheptyl.

As used herein the term “heterocyclyl,” by itself or as part of another substituent, refers to a saturated ring system having from 3 to 12 ring members and from 1 to 4 heteroatoms of N, O and S. Additional heteroatoms can also be useful, including, but not limited to, B, Al, Si and P. The heteroatoms can be oxidized to form moieties such as, but not limited to, —S(O)— and —S(O)₂—. Heterocyclyl groups can include any number of ring atoms, such as, C₃₋₆, C₄₋₆, C₅₋₆, C₃₋₈, C₄₋₈, C₅₋₈, C₆₋₈, C₃₋₉, C₃₋₁₀, C₃₋₁₁, or C₃₋₁₂, wherein at least one of the carbon atoms is replaced by a heteroatom. Any suitable number of carbon ring atoms can be replaced with heteroatoms in the heterocyclyl groups, such as 1, 2, 3, or 4, or 1 to 2, 1 to 3, 1 to 4, 2 to 3, 2 to 4, or 3 to 4. The heterocyclyl group can include groups such as aziridine, azetidine, pyrrolidine, piperidine, azepane, azocane, quinuclidine, pyrazolidine, imidazolidine, piperazine (1,2-, 1,3- and 1,4-isomers), oxirane, oxetane, tetrahydrofuran, oxane (tetrahydropyran), oxepane, thiirane, thietane, thiolane (tetrahydrothiophene), thiane (tetrahydrothiopyran), oxazolidine, isoxazolidine, thiazolidine, isothiazolidine, dioxolane, dithiolane, morpholine, thiomorpholine, dioxane, or dithiane. The heterocyclyl groups can also be fused to aromatic or non-aromatic ring systems to form members including, but not limited to, indoline. Heterocyclyl groups can be unsubstituted or substituted. Unless otherwise specified, “substituted heterocyclyl” groups can be substituted with one or more groups selected from halo, hydroxy, amino, oxo (═O), alkylamino, amido, acyl, nitro, cyano, and alkoxy.

The heterocyclyl groups can be linked via any position on the ring. For example, aziridine can be 1- or 2-aziridine, azetidine can be 1- or 2-azetidine, pyrrolidine can be 1-, 2- or 3-pyrrolidine, piperidine can be 1-, 2-, 3- or 4-piperidine, pyrazolidine can be 1-, 2-, 3-, or 4-pyrazolidine, imidazolidine can be 1-, 2-, 3- or 4-imidazolidine, piperazine can be 1-, 2-, 3- or 4-piperazine, tetrahydrofuran can be 1- or 2-tetrahydrofuran, oxazolidine can be 2-, 3-, 4- or 5-oxazolidine, isoxazolidine can be 2-, 3-, 4- or 5-isoxazolidine, thiazolidine can be 2-, 3-, 4- or 5-thiazolidine, isothiazolidine can be 2-, 3-, 4- or 5-isothiazolidine, and morpholine can be 2-, 3- or 4-morpholine.

When heterocyclyl includes 3 to 8 ring members and 1 to 3 heteroatoms, representative members include, but are not limited to, pyrrolidine, piperidine, tetrahydrofuran, oxane, tetrahydrothiophene, thiane, pyrazolidine, imidazolidine, piperazine, oxazolidine, isoxazolidine, thiazolidine, isothiazolidine, morpholine, thiomorpholine, dioxane and dithiane. Heterocyclyl can also form a ring having 5 to 6 ring members and 1 to 2 heteroatoms, with representative members including, but not limited to, pyrrolidine, piperidine, tetrahydrofuran, tetrahydrothiophene, pyrazolidine, imidazolidine, piperazine, oxazolidine, isoxazolidine, thiazolidine, isothiazolidine, and morpholine.

As used herein the term “aryl,” by itself or as part of another substituent, refers to an aromatic ring system having any suitable number of carbon ring atoms and any suitable number of rings. Aryl groups can include any suitable number of carbon ring atoms, such as C₆, C₇, C₈, C₉, C₁₀, C₁₁, C₁₂, C₁₃, C₁₄, C₁₅ or C₁₆, as well as C₆₋₁₀, C₆₋₁₂, or C₆₋₁₄. Aryl groups can be monocyclic, fused to form bicyclic (e.g., benzocyclohexyl) or tricyclic groups, or linked by a bond to form a biaryl group. Representative aryl groups include phenyl, naphthyl and biphenyl. Other aryl groups include benzyl, having a methylene linking group. Some aryl groups have from 6 to 12 ring members, such as phenyl, naphthyl or biphenyl. Other aryl groups have from 6 to 10 ring members, such as phenyl or naphthyl. Some other aryl groups have 6 ring members, such as phenyl. Aryl groups can be substituted or unsubstituted. Unless otherwise specified, “substituted aryl” groups can be substituted with one or more groups selected from halo, hydroxy, amino, alkylamino, amido, acyl, nitro, cyano, and alkoxy.

As used herein the term “heteroaryl,” by itself or as part of another substituent, refers to a monocyclic or fused bicyclic or tricyclic aromatic ring assembly containing 5 to 16 ring atoms, where from 1 to 5 of the ring atoms are a heteroatom such as N, O or S. Additional heteroatoms can also be useful, including, but not limited to, B, Al, Si and P. The heteroatoms can be oxidized to form moieties such as, but not limited to, —S(O)— and —S(O)₂—. Heteroaryl groups can include any number of ring atoms, such as C₅₋₆, C₃₋₈, C₄₋₈, C₅₋₈, C₆₋₈, C₃₋₉, C₃₋₁₀, C₃₋₁₁, or C₃₋₁₂, wherein at least one of the carbon atoms is replaced by a heteroatom. Any suitable number of heteroatoms can be included in the heteroaryl groups, such as 1, 2, 3, 4; or 5, or 1 to 2, 1 to 3, 1 to 4, 1 to 5, 2 to 3, 2 to 4, 2 to 5, 3 to 4, or 3 to 5. For example, heteroaryl groups can be C₅₋₈ heteroaryl, wherein 1 to 4 carbon ring atoms are replaced with heteroatoms; or C₅₋₈ heteroaryl, wherein 1 to 3 carbon ring atoms are replaced with heteroatoms; or C₅₋₆ heteroaryl, wherein 1 to 4 carbon ring atoms are replaced with heteroatoms; or C₅₋₆ heteroaryl, wherein 1 to 3 carbon ring atoms are replaced with heteroatoms. The heteroaryl group can include groups such as pyrrole, pyridine, imidazole, pyrazole, triazole, tetrazole, pyrazine, pyrimidine, pyridazine, triazine (1,2,3-, 1,2,4- and 1,3,5-isomers), thiophene, furan, thiazole, isothiazole, oxazole, and isoxazole. The heteroaryl groups can also be fused to aromatic ring systems, such as a phenyl ring, to form members including, but not limited to, benzopyrroles such as indole and isoindole, benzopyridines such as quinoline and isoquinoline, benzopyrazine (quinoxaline), benzopyrimidine (quinazoline), benzopyridazines such as phthalazine and cinnoline, benzothiophene, and benzofuran. Other heteroaryl groups include heteroaryl rings linked by a bond, such as bipyridine. Heteroaryl groups can be substituted or unsubstituted. Unless otherwise specified, “substituted heteroaryl” groups can be substituted with one or more groups selected from halo, hydroxy, amino, alkylamino, amido, acyl, nitro, cyano, and alkoxy.

The heteroaryl groups can be linked via any position on the ring. For example, pyrrole includes 1-, 2- and 3-pyrrole, pyridine includes 2-, 3- and 4-pyridine, imidazole includes 1-, 2-, 4- and 5-imidazole, pyrazole includes 1-, 3-, 4- and 5-pyrazole, triazole includes 1-, 4- and 5-triazole, tetrazole includes 1- and 5-tetrazole, pyrimidine includes 2-, 4-, 5- and 6-pyrimidine, pyridazine includes 3- and 4-pyridazine, 1,2,3-triazine includes 4- and 5-triazine, 1,2,4-triazine includes 3-, 5- and 6-triazine, 1,3,5-triazine includes 2-triazine, thiophene includes 2- and 3-thiophene, furan includes 2- and 3-furan, thiazole includes 2-, 4- and 5-thiazole, isothiazole includes 3-, 4- and 5-isothiazole, oxazole includes 2-, 4- and 5-oxazole, isoxazole includes 3-, 4- and 5-isoxazole, indole includes 1-, 2- and 3-indole, isoindole includes 1- and 2-isoindole, quinoline includes 2-, 3- and 4-quinoline, isoquinoline includes 1-, 3- and 4-isoquinoline, quinazoline includes 2- and 4-quinazoline, cinnoline includes 3- and 4-cinnoline, benzothiophene includes 2- and 3-benzothiophene, and benzofuran includes 2- and 3-benzofuran.

Some heteroaryl groups include those having from 5 to 10 ring members and from 1 to 3 ring atoms including N, O or S, such as pyrrole, pyridine, imidazole, pyrazole, triazole, pyrazine, pyrimidine, pyridazine, triazine (1,2,3-, 1,2,4- and 1,3,5-isomers), thiophene, furan, thiazole, isothiazole, oxazole, isoxazole, indole, isoindole, quinoline, isoquinoline, quinoxaline, quinazoline, phthalazine, cinnoline, benzothiophene, and benzofuran. Other heteroaryl groups include those having from 5 to 8 ring members and from 1 to 3 heteroatoms, such as pyrrole, pyridine, imidazole, pyrazole, triazole, pyrazine, pyrimidine, pyridazine, triazine (1,2,3-, 1,2,4- and 1,3,5-isomers), thiophene, furan, thiazole, isothiazole, oxazole, and isoxazole. Some other heteroaryl groups include those having from 9 to 12 ring members and from 1 to 3 heteroatoms, such as indole, isoindole, quinoline, isoquinoline, quinoxaline, quinazoline, phthalazine, cinnoline, benzothiophene, benzofuran and bipyridine. Still other heteroaryl groups include those having from 5 to 6 ring members and from 1 to 2 ring atoms including N, O or S, such as pyrrole, pyridine, imidazole, pyrazole, pyrazine, pyrimidine, pyridazine, thiophene, furan, thiazole, isothiazole, oxazole, and isoxazole.

Some heteroaryl groups include from 5 to 10 ring members and only nitrogen heteroatoms, such as pyrrole, pyridine, imidazole, pyrazole, triazole, pyrazine, pyrimidine, pyridazine, triazine (1,2,3-, 1,2,4- and 1,3,5-isomers), indole, isoindole, quinoline, isoquinoline, quinoxaline, quinazoline, phthalazine, and cinnoline. Other heteroaryl groups include from 5 to 10 ring members and only oxygen heteroatoms, such as furan and benzofuran. Some other heteroaryl groups include from 5 to 10 ring members and only sulfur heteroatoms, such as thiophene and benzothiophene. Still other heteroaryl groups include from 5 to 10 ring members and at least two heteroatoms, such as imidazole, pyrazole, triazole, pyrazine, pyrimidine, pyridazine, triazine (1,2,3-, 1,2,4- and 1,3,5-isomers), thiazole, isothiazole, oxazole, isoxazole, quinoxaline, quinazoline, phthalazine, and cinnoline.

As used herein, the terms “halo” and “halogen,” by themselves or as part of another substituent, refer to a fluorine, chlorine, bromine, or iodine atom.

As used herein, the term “hydroxy” refers to the moiety —OH.

As used herein, the term “amino” refers to a moiety —NR₂, wherein each R group is H or alkyl. An amino moiety can be ionized to form the corresponding ammonium cation.

As used herein, the term “amido” refers to a moiety —NRC(O)R or —C(O)NR₂, wherein each R group is H or alkyl.

As used herein, the term “acyl” refers to a moiety —C(O)R, wherein R is H or alkyl.

As used herein, the term “nitro” refers to the moiety —NO₂.

As used herein, the term “cyano” refers to a carbon atom triple-bonded to a nitrogen atom (i.e., the moiety —C≡N).

As used herein, the terms “molar percentage” and “mol %” refer to the number of a moles of a given lipid component of a liposome divided by the total number of moles of all lipid components. Unless explicitly stated, the amounts of active agents, diluents, or other components are not included when calculating the mol % for a lipid component of a liposome.

As used herein, the term “therapeutic agent” refers to a compound or molecule that, when present in an effective amount, produces a desired therapeutic effect in a subject in need thereof. The present disclosure contemplates a broad range of therapeutic agents and their use in conjunction with the liposome compositions. As used herein, the term “subject” refers to any mammal, in particular a human, at any stage of life.

As used herein, the term “diagnostic agent” refers to a molecule which can be directly or indirectly detected and is used for diagnostic purposes. According to some embodiments of the invention, the diagnosis is effected in vivo (i.e., within a living subject) using e.g., MRI, CT, radiography and fluoroscopy.

As used herein, the term “targeting agent” refers to a molecule that is specific for a target, such as a organs, tissues, cells, extracellular matrix components, and/or intracellular compartments that can be associated with a specific developmental stage of a disease. Targets can further include antigens on a surface of a cell, or a tumor marker that is an antigen present or more prevalent on a cancer cell as compared to normal tissue. Examples of targeting agents include, but are not limited to, antibodies and nucleic acid aptamers.

As used herein, the term “antibody” refers to a polypeptide comprising an antigen binding region (including the complementarity determining region (CDRs)) from an immunoglobulin gene or fragments thereof that specifically binds and recognizes an antigen. The recognized immunoglobulin genes include the kappa, lambda, alpha, gamma, delta, epsilon, and mu constant region genes, as well as numerous immunoglobulin variable region genes.

As used herein, the term “consists essentially of” refers to a composition having the stated components, in addition to minor components (e.g., unavoidable impurities) that do not materially affect the properties of the composition (e.g., the average size or monodispersity of a population of liposomes).

As used herein, the term “about” indicates a close range around a numerical value when used to modify that specific value. If “X” were the value, for example, “about X” would indicate a value from 0.9X to 1.1X, e.g., a value from 0.95X to 1.05X, or a value from 0.98X to 1.02X, or a value from 0.99X to 1.01X. Any reference to “about X” specifically indicates at least the values X, 0.9X, 0.91X, 0.92X, 0.93X, 0.94X, 0.95X, 0.96X, 0.97X, 0.98X, 0.99X, 1.01X, 1.02X, 1.03X, 1.04X, 1.05X, 1.06X, 1.07X, 1.08X, 1.09X, and 1.1X, and values within this range.

### III. MULTIPLEXED PARTICLES

Provided herein is a particle comprising a first effector element covalently bonded to a nanoparticle via a first orthogonal moiety and a second effector element covalently bonded to the nanoparticle via a second orthogonal moiety, wherein the first orthogonal moiety and the second orthogonal moiety have different chemical structures. In some embodiments, the first effector element and the second effector element are independently selected from the group consisting of a targeting agent, a therapeutic agent, and a diagnostic agent.

### Bioorthogonal Functionalization

In general, an orthogonal moiety in a particle (e.g., a first orthogonal moiety, a second orthogonal moiety, or an additional orthogonal moiety) is the product of a reaction between a pair of reaction partners, i.e., a reactive functional group and a reactive effector element. Each of the reaction partners is typically reactive with its counterpart in the orthogonal pair, but it is typically unreactive (or reversibly reactive) with other molecules. The reaction between orthogonal reaction partners, often referred to as “click reactions,” are typically characterized by a large thermodynamic driving force that usually results in irreversible covalent bond formation. In certain instances, the orthogonal pair is a “bioorthogonal” pair, meaning that the reaction partners are generally unreactive (or reversibly reactive) with biological nucleophiles such as amines, thiols, and alcohols and other functional groups in the biological milieu. The reactions can often be conducted in aqueous or physiological conditions without producing cytotoxic byproducts. A number of such bioorthogonal reactive pairs can be used for preparing the particles disclosed herein. Examples of bioorthogonal reactive pairs include, but are not limited to: ketones/aldehydes and aminooxy compounds/hydrazides; azides and alkynes, including cyclooctynes; azides and phosphines, including triarylphosphines; tetrazines and alkenes/alkynes, including trans-cyclooctenes, norbornenes, and cyclooctynes; and cyclopentadienones and alkynes, including cyclooctynes.

In some embodiments, the first orthogonal moiety comprises a dihydropyridazine, a pyridazine, a triazole, a hydrazide, an oxime, a phosphoryl-substituted amide, or a thioether.

In some embodiments, the first orthogonal moiety comprises a dihydropyridazine according to Formula Ia

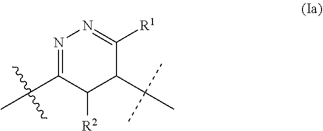

wherein
the wavy line represents the point of connection to the particle,
the dashed line represents the point of connection to the effector element,
R¹ is selected from the group consisting of H and C₁₋₆ alkyl, and
R² is selected from the group consisting of H, C₁₋₆ alkyl, C₃₋₈ cycloalkyl, 5-to-12-membered heterocyclyl, C₆₋₁₀ aryl, and 5-to-12-membered heteroaryl,
or, alternatively, R² is taken together with the point of connection to the effector element to form a fused bicyclic group or a fused tricyclic group.

The monocyclic group, the fused bicyclic group, and fused the tricyclic group can be a carbocyclic group or a heterocyclic group. The “point of attachment” to either the particle or the effector element can be a direct attachment (i.e., a covalent bond) or an attachment via one or more intervening atoms (e.g., a linking moiety as described in more detail below).

In some embodiments, the first orthogonal moiety comprises a dihydropyridazine according to Formula Ib

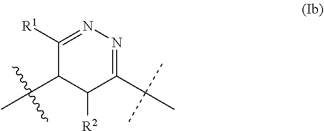

wherein
the wavy line represents the point of connection to the particle,
the dashed line represents the point of connection to the effector element,
R¹ is selected from the group consisting of H and C₁₋₆ alkyl,
R² is selected from the group consisting of H, C₁₋₆ alkyl, C₃₋₈ cycloalkyl, 5-to-12-membered heterocyclyl, C₆₋₁₀ aryl, and 5-to-12-membered heteroaryl,
or, alternatively, R² is taken together with the point of connection to the particle to form a monocyclic group, a fused bicyclic group, or a fused tricyclic group.

In some embodiments, R² is taken together with the point of attachment to the effector element to form a monocyclic group such as a cycloalkyl group (e.g., a cycloctane group), which is fused to the dihydropyridazine core in Formula Ia. The fused cycloalkyl group can be bonded to the effector element directly (i.e., by a covalent bond) or via one or more intervening atoms (e.g., a linking moiety as described below).

In some embodiments, R² is taken together with the point of attachment to the particle to form a monocyclic group such as a cycloalkyl group (e.g., a cycloctane group), which is fused to the dihydropyridazine core in Formula Ib. The fused cycloalkyl group can be bonded to the particle directly (i.e., by a covalent bond) or via one or more intervening atoms (e.g., a linking moiety as described below).

In some embodiments, the first orthogonal moiety comprises a dihydropyridazine according to Formula Ic.

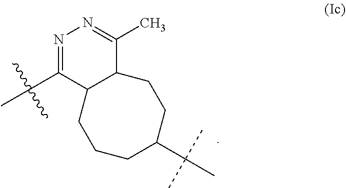

In some embodiments, the first orthogonal moiety comprises a pyridazine according to Formula IIa:

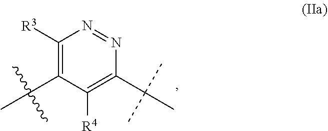

wherein
the wavy line represents the point of connection to the particle,
the dashed line represents the point of connection to the effector element,
R³ is selected from the group consisting of H and C₁₋₆ alkyl, and
R⁴ is selected from the group consisting of H, C₁₋₆ alkyl, C₃₋₈ cycloalkyl, 5-to-12-membered heterocyclyl, C₆₋₁₀ aryl, and 5-to-12-membered heteroaryl,
or, alternatively, R⁴ is taken together with the point of connection to the particle to form a monocyclic group, a fused bicyclic group, or a fused tricyclic group.

In some embodiments, the first orthogonal moiety comprises a pyridazine according to Formula IIb

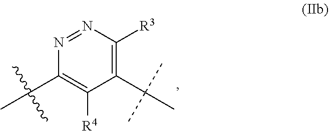

wherein
the wavy line represents the point of connection to the particle,
the dashed line represents the point of connection to the effector element,
R³ is selected from the group consisting of H and C₁₋₆ alkyl,
R⁴ is selected from the group consisting of H, C₁₋₆ alkyl, C₃₋₈ cycloalkyl, 5-to-12-membered heterocyclyl, C₆₋₁₀ aryl, and 5-to-12-membered heteroaryl,
or, alternatively, R⁴ is taken together with the point of connection to the effector element to form a fused bicyclic group or a fused tricyclic group.

In some embodiments, R⁴ is taken together with the point of attachment to the particle to form a monocyclic group such as a monoheterocycle (e.g., an azocane group) or fused tricyclic group (e.g., a dibenzo-fused tetrahydroazocine group), which is fused to the pyridazine core in Formula IIa. The monoheterocycle or the fused tricyclic group can be bonded to the particle directly (i.e., by a covalent bond) or via one or more intervening atoms (e.g., a linking moiety as described below).

In some embodiments, R⁴ is taken together with the point of attachment to the effector element to form a monocyclic group such as a monoheterocycle (e.g., an azocane group) or fused tricyclic group (e.g., a dibenzo-fused tetrahydroazocine group), which is fused to the pyridazine core in Formula IIb. The monoheterocycle or the fused tricyclic group can be bonded to the effector element directly (i.e., by a covalent bond) or via one or more intervening atoms (e.g., a linking moiety as described below).

In some embodiments, the first orthogonal moiety comprises a triazole. In some embodiments, the first orthogonal moiety comprises a triazole according to Formula IIIa:

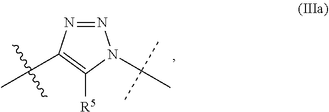

wherein
the wavy line represents the point of connection to the particle,
the dashed line represents the point of connection to the effector element,
R⁵ is selected from the group consisting of H, C₁₋₆ alkyl, C₃₋₈ cycloalkyl, 5-to-12-membered heterocyclyl, C₆₋₁₀ aryl, and 5-to-12-membered heteroaryl,
or, alternatively, R⁵ is taken together with the point of connection to the particle to form a monocyclic group, a fused bicyclic group, or a fused tricyclic group.

In some embodiments, the first orthogonal moiety comprises a triazole. In some embodiments, the first orthogonal moiety comprises a triazole according to Formula IIIb:

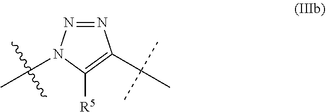

wherein
the wavy line represents the point of connection to the particle,
the dashed line represents the point of connection to the effector element,
R⁵ is selected from the group consisting of H, C₁₋₆ alkyl, C₃₋₈ cycloalkyl, 5-to-12-membered heterocyclyl, C₆₋₁₀ aryl, and 5-to-12-membered heteroaryl,
or, alternatively, R⁵ is taken together with the point of connection to the effector element to form a monocyclic group, a fused bicyclic group, or a fused tricyclic group.

In some embodiments, R⁵ is taken together with the point of attachment to the particle to form a monocyclic group such as a monoheterocycle (e.g., an azocane group) or fused tricyclic group (e.g., a dibenzo-fused tetrahydroazocine group), which is fused to the triazole core in Formula IIIa. The monoheterocycle or the fused tricyclic group can be bonded to the particle directly (i.e., by a covalent bond) or via one or more intervening atoms (e.g., a linking moiety as described below).

In some embodiments, R⁵ is taken together with the point of attachment to the effector element to form a monocyclic group such as a monoheterocycle (e.g., an azocane group) or fused tricyclic group (e.g., a dibenzo-fused tetrahydroazocine group), which is fused to the triazole core in Formula IIIb. The monoheterocycle or the fused tricyclic group can be bonded to the effector element directly (i.e., by a covalent bond) or via one or more intervening atoms (e.g., a linking moiety as described below).

In some embodiments, the first orthogonal moiety comprises a triazole according to Formula IIIc

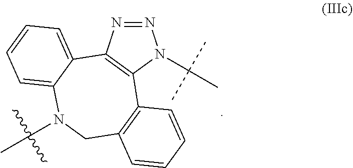

In some embodiments, the first orthogonal moiety comprises a hydrazide. In some embodiments, the first orthogonal moiety comprises a hydrazide according to Formula IVa or Formula IVb i.e.,

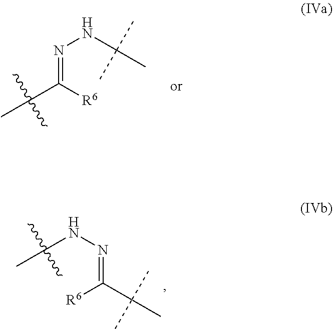

wherein
the wavy line represents the point of connection to the particle,
the dashed line represents the point of connection to the effector element, and
R⁶ is selected from the group consisting of H, C₁₋₆ alkyl, C₃₋₈ cycloalkyl, 5-to-12-membered heterocyclyl, C₆₋₁₀ aryl, and 5-to-12-membered heteroaryl.

In some embodiments, the first orthogonal moiety comprises an oxime. In some embodiments, the first orthogonal moiety comprises an oxime according to Formula Va or Formula Vb, i.e.,

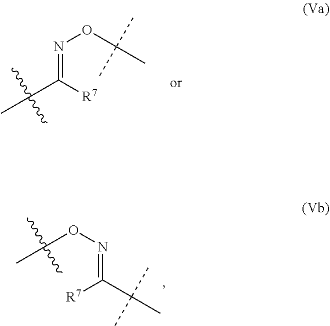

wherein
the wavy line represents the point of connection to the particle,
the dashed line represents the point of connection to the effector element, and
R⁶ is selected from the group consisting of H, C₁₋₆ alkyl, C₃₋₈ cycloalkyl, 5-to-12-membered heterocyclyl, C₆₋₁₀ aryl, and 5-to-12-membered heteroaryl.

In some embodiments, the first orthogonal moiety comprises a phosphoryl-substituted amide. In some embodiments, the first orthogonal moiety comprises a phosphoryl-substituted amide according to Formula VIa or Formula VIb, i.e.,

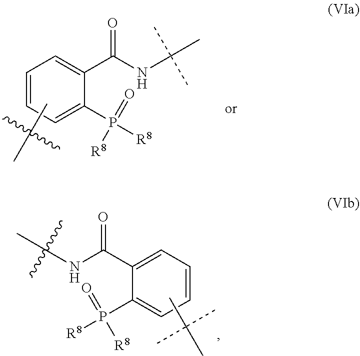

wherein
the wavy line represents the point of connection to the particle,
the dashed line represents the point of connection to the effector element, and
each R⁸ is independently selected from the group consisting of C₁₋₆ alkyl, C₃₋₈ cycloalkyl, 5-to-12-membered heterocyclyl, C₆₋₁₀ aryl, and 5-to-12-membered heteroaryl.

In some embodiments, the first orthogonal moiety comprises a thioether. In some embodiments, the first orthogonal moiety comprises a thioether according to Formula VIIa or Formula VIIb, i.e.,

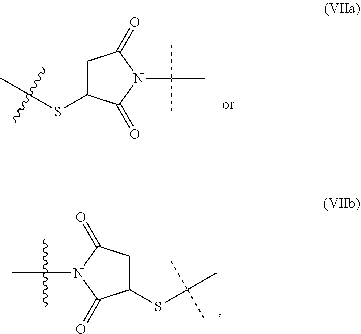

wherein
the wavy line represents the point of connection to the particle, and
the dashed line represents the point of connection to the effector element.

The orthogonal moieties of the particles disclosed herein may be optionally substituted with further functional groups. In general, the term “substituted,” whether preceded by the term “optionally” or not, means that one or more hydrogen atoms in a designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents are generally those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein. In general, “substituted,” as used herein, does not encompass replacement and/or alteration of a key functional group by which a molecule is identified, e.g., such that the “substituted” functional group becomes, through substitution, a different functional group. For example, a “substituted phenyl” group must still comprise the phenyl moiety and cannot be modified by substitution, in this definition, to become, e.g., a cyclohexyl group.

Suitable monovalent substituents on a substitutable carbon atom of an “optionally substituted” group are independently halogen; —(CH₂)₀₋₄R^{α}; —(CH₂)₀₋₄R^{α}; —O(CH₂)₀₋₄R^{α}, —O—(CH₂)₀₋₄C(O)OR^{α}; —(CH₂)₀₋₄CH(OR^{α})₂; —(CH₂)₀₋₄SR^{α}; —(CH₂)₀₋₄Ph, wherein Ph is phenyl which may be substituted with R^{α}; —(CH₂)₀₋₄O(CH₂)₀₋₁phenyl, which phenyl may be substituted with R^{α}; —CH═CHPh, wherein Ph is phenyl which may be substituted with R^{α}; —(CH₂)₀₋₄O(CH₂)₀₋₁-Py, wherein Py is pyridyl which may be substituted with R^{α}; —NO₂: —CN; —N₃; —(CH₂)₀₋₄N(R^{α})₂; —(CH₂)₀₋₄N(R^{α})C(O)R^{α}; —N(R^{α})C(S)R^{α}; —(CH₂)₀₋₄N(R^{α})C(O)NR^{α}₂; —N(R^{α})C(S)NR^{α}₂; —(CH₂)₀₋₄N(R^{α})C(O)OR^{α}; —N(R^{α})N(R^{α})C(O)R^{α}; —N(R^{α})N(R^{α})C(O)NR^{α}₂; —N(R^{α})N(R^{α})C(O)OR^{α}; —(CH₂)₀₋₄C(O)R^{α}; —C(S)R^{α}; —(CH₂)₀₋₄C(O)OR^{α}; —(CH₂)₀₋₄C(O)SR^{α}; —(CH₂)₀₋₄C(O)OSiR^{α}₃; —(CH₂)₀₋₄OC(O)R^{α}; —OC(O)(CH₂)₀₋₄SR—SC(S)SR^{α}; —(CH₂)₀₋₄SC(O)R^{α}; —(CH₂)₀₋₄C(O)NR^{α}₂; —C(S)NR^{α}₂, —C(S)SR^{α}; —SC(S)SR^{α}, —(CH₂)₀₋₄OC(O)NR^{α}₂; —C(O)N(OR^{α})R^{α}; —C(O)C(O)R^{α}; —C(O)CH₂C(O)R^{α}; —C(NOR^{α})R^{α}; —(CH₂)₀₋₄SSR^{α}; —(CH₂)₀₋₄S(O)₂NR^{α}, —(CH₂)₀₋₄S(O)₂OR^{α}; —(CH₂)₀₋₄OS(O)₂R^{α}; —S(O)₂NR^{α}₂; —(CH₂)₀₋₄S(O)R^{α}; —N(R^{α})S(O)₂NR^{α}₂; —N(R^{α})S(O)₂R^{α}; —N(OR^{α})R^{α}; —C(NH)NR^{α}₂; —P(O)₂R^{α}, —P(O)R^{α}₂; —OP(O)R^{α}₂; —OP(O)(OR^{α})₂; SiR^{α}₃; —(C₁₋₄ straight or branched)alkylene)O—N(R^{α})₂; or —(C₁₋₄ straight or branched)alkylene)C(O)O—N(R^{α})₂. Each R^{α} is independently hydrogen; C₁₋₆ alkyl; —CH₂Ph, —O(CH₂)₀₋₁Ph; —CH₂-(5- to 6-membered heteroaryl); C₃₋₈ cycloalkyl; C₆₋₁₀ aryl; 4- to 10-membered heterocyclyl; or 6- to 10-membered heteroaryl; and each R^{α} may be further substituted as described below.

Suitable monovalent substituents on R^{α} are independently halogen, —(CH₂)₀₋₂R^{β}; —(CH₂)₀₋₂OH; —(CH₂)₀₋₂OR^{β}; —(CH₂)₀₋₂CH(OR^{β})₂, —CN; —N₃; —(CH₂)₀₋₂C(O)R^{β}; —(CH₂)₀₋₂C(O)OH; —(CH₂)₀₋₂C(O)OR^{β}; —(CH₂)₀₋₂SR^{β}; —(CH₂)₀₋₂SH; —(CH₂)₀₋₂NH₂; —(CH₂)₀₋₂NHR^{β}; —(CH₂)₀₋₂NR^{β}₂; —NO₂; SiR^{β}₃; —OSiR^{β}₃; —C(O)SR^{β}; —(C₁₋₄ straight or branched alkylene)C(O)OR^{β}; or —SSR^{β}; wherein each R^{β} e is independently selected from C₁₋₄ alkyl; —CH₂Ph; —O(CH₂)₀₋₁Ph; C₃₋₈ cycloalkyl; C₆₋₁₀ aryl; 4- to 10-membered heterocyclyl; or 6- to 10-membered heteroaryl. Suitable divalent substituents on a saturated carbon atom of R^{α} include ═O and ═S.

Suitable divalent substituents on a saturated carbon atom of an “optionally substituted” group include the following: ═O; ═S; ═NNR^{γ}₂; ═NNHC(O)R^{γ}; ═NNHC(O)OR^{γ}; ═NNHS(O)₂R^{γ}; αNR^{γ}; ═NOR^{γ}; —O(C(R^{γ}₂))₂₋₃O—; or —S(C(R^{γ}₂))₂₋₃S—; wherein each independent occurrence of R^{γ} is selected from hydrogen; C₁₋₆ alkyl, which may be substituted as defined below; C₃₋₈ cycloalkyl; C₆₋₁₀ aryl; 4- to 10-membered heterocyclyl; or 6- to 10-membered heteroaryl. Suitable divalent substituents that are bound to vicinal substitutable carbons of an “optionally substituted” group include: —O(CR^{β}₂)₂₋₃O—; wherein each independent occurrence of R^{β} is selected from hydrogen; C₁₋₆ alkyl which may be substituted as defined below; C₃₋₈ cycloalkyl; C₆₋₁₀ aryl; 4- to 10-membered heterocyclyl; or 6- to 10-membered heteroaryl.

Suitable substituents on the alkyl group of R^{γ} include halogen; —R^{δ}; —OH; —OR^{δ}; —CN; —C(O)OH; —C(O)OR^{δ}; —NH₂; —NHR^{δ}; —NR^{δ}₂; or —NO₂; wherein each R^{δ} is independently C₁₋₄ alkyl; —CH₂Ph; —O(CH₂)₀₋₁Ph; 4- to 10-membered heterocyclyl; or 6- to 10-membered heteroaryl.

Suitable substituents on a substitutable nitrogen of an “optionally substituted” group include —R^{ε}; —NR^{ε}₂; —C(O)R^{ε}; —C(O)OR^{ε}; —C(O)C(O)R^{ε}; —C(O)CH₂C(O)R^{ε}; —S(O)₂R^{ε}; —S(O)₂NR^{ε}₂; —C(S)NR^{ε}₂; —C(NH)NR^{ε}₂; or —N(R^{ε})S(O)₂R^{ε}; wherein each R^{ε} is independently hydrogen; C₁₋₆ alkyl which may be substituted as defined below; C₃₋₈ cycloalkyl; C₆₋₁₀ aryl; 4- to 10-membered heterocyclyl; or 6- to 10-membered heteroaryl.

Suitable substituents on the alkyl group of W are independently halogen; —R^{δ}; —OH; —OR^{δ}; —CN; —C(O)OH; —C(O)OR^{δ}; —NH₂; —NHR^{δ}; —NR^{δ}₂; or —NO₂; wherein each R^{δ} is independently C₁₋₄ alkyl; —CH₂Ph; —O(CH₂)₀₋₁Ph; C₆₋₁₀ aryl; 4- to 10-membered heterocyclyl; or 6- to 10-membered heteroaryl.

As set forth above, an orthogonal moiety is covalently attached to a particle and an effector element by a covalent bond or via one or more intervening atoms. In certain embodiments, the particle contains a first linking moiety between the first orthogonal moiety and the nanoparticle. In some such embodiments, the particle also contains a second linking moiety between the second orthogonal moiety and the effector element. A number of suitable linkers can be used for preparing the particles provided herein. In general, the structure of the linker (e.g., the length of the linker, functional groups present in the linker, etc.) is designed to reduce or eliminate steric hindrance which would otherwise prevent formation of the orthogonal moiety. The structure of the linker can also be designed to ensure that the effector element (e.g., a targeting agent) is not prevented from performing its intended function (e.g., recognizing its intended target) upon attachment to the nanoparticle. Examples of linking moieties include, but are not limited to, cleavable and non-cleavable linking moieties as described, for example, in Hermanson, Bioconjugate Techniques 2nd Edition, Academic Press, 2008.

In some embodiments, the linking moiety L¹ has a structure -L^{1a}-L^{1b}, wherein L^{1a} and L^{1b} are independently selected from a bond, a divalent polymer moiety, and linear or branched, saturated or unsaturated C₁₋₃₀ alkylene;

wherein one or more non-adjacent carbon atoms in the C₁₋₃₀ alkylene are optionally and independently replaced by O, S, NR^{a};

wherein one or more groupings of adjacent carbon atoms in the C₁₋₃₀ alkylene are optionally and independently replaced by —NR^{a}(CO)— or —(CO)NR^{a}—; and

wherein one or more groupings of adjacent carbon atoms in the C₁₋₃₀ alkylene are optionally and independently replaced by a 4- to 8-membered, divalent carbocycle or a 4- to 8-membered, divalent heterocycle having one to four heteroatoms selected from O, S, and N; and

wherein each R^{a} is independently selected from H and C₁₋₆ alkyl.

A divalent polymer moiety in a linking moiety L may contain functional groups at one or both ends of the polymer chain. As a non-limiting example, one end of a divalent polymer may contain a carbonyl group (i.e., C═O) which may be part of an amide linkage or a carbamate linkage connecting the divalent polymer to a nitrogen atom present on a particle. Accordingly, some embodiments provide particles having a first orthogonal moiety according to Formula Id

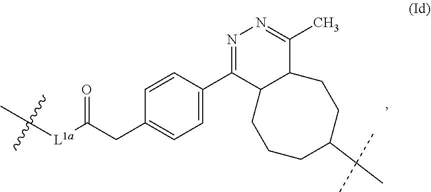

wherein L^{1a} is as described above. In some embodiments, L^{1a} is a divalent polymer moiety.

In some embodiments, the particle contains a first orthogonal moiety according to Formula IIId:

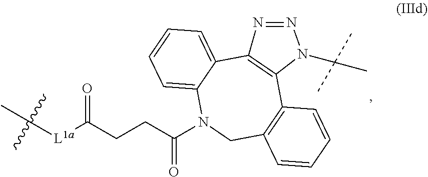

wherein L^{1a} is as described above. In some embodiments, L^{1a} is a divalent polymer moiety.

In some embodiments, the first linking moiety comprises an oligo(ethylene glycol) or a poly(ethylene glycol). Oligo(ethylene glycol) and poly(ethylene glycol) of any suitable molecular weight can be included in the linking moieties. For example, an oligo(ethylene glycol) may contain between 2 and 10 repeating ethylene glycol units (i.e., units with the structure —CH₂CH₂O—). The oligo(ethylene glycol) can contain about 2-4, 2-6, 2-8, 4-6, 4-8, 4-10, 6-8, 6-10, or 8-10 repeating ethylene glycol units. A poly(ethylene glycol) may range in molecular weight, for example, from about 500 Da to about 10,000 Da. In some embodiments, the oligo(ethylene glycol) contains about 4 repeating ethylene glycol units.

The molecular of the poly(ethylene glycol) can range from about 500 Da to about 1000 Da, or from about 1000 Da to about 2000 Da, or from about 2000 Da to about 3000 Da, or from about 3000 Da to about 4000 Da, or from about 4000 Da to about 5000 Da, or from about 5000 Da to about 6000 Da, or from about 6000 Da, or from about 6000 Da to about 7000 Da, or from about 8000 Da to about 9000 Da, or from about 9000 Da to about 10,000 Da. The molecular of the poly(ethylene glycol) can range from about 1000 Da to about 10,000 Da, or from about 2000 Da to about 8000 Da, or from about 4000 Da to about 6000 Da. In some embodiments, the molecular weight of the poly(ethylene glycol) is about 2000 Da. In some embodiments, the molecular weight of the poly(ethylene glycol) is about 5000 Da.

The particles disclosed herein may also contain a linking moiety connecting the first orthogonal moiety to the first effector element. In some such embodiments, a linking moiety -L^{2a}-L^{2b}- connects the first orthogonal moiety to the first effector element, L^{2a} and L^{2b} are independently selected from a bond, a divalent polymer moiety, and linear or branched, saturated or unsaturated C₁₋₃₀ alkylene;

wherein one or more non-adjacent carbon atoms in the C₁₋₃₀ alkylene are optionally and independently replaced by O, S, NR^{a};

wherein one or more groupings of adjacent carbon atoms in the C₁₋₃₀ alkylene are optionally and independently replaced by —NR^{a}(CO)— or —(CO)NR^{a}—; and

wherein one or more groupings of adjacent carbon atoms in the C₁₋₃₀ alkylene are optionally and independently replaced by a 4- to 8-membered, divalent carbocycle or a 4- to 8-membered, divalent heterocycle having one to four heteroatoms selected from O, S, and N; and

wherein each R^{a} is independently selected from H and C₁₋₆ alkyl.

In some embodiments, the particle contains a first orthogonal moiety according to Formula Ie:

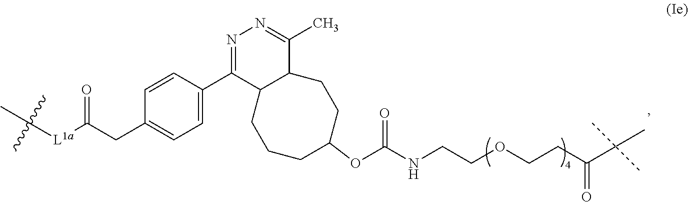

wherein L^{1a} is as described above. In some embodiments, L^{1a} is a divalent polymer moiety.

In addition to the first orthogonal moiety described above, the particles provided herein also contain one or more additional orthogonal moieties (e.g., a second orthogonal moiety; second and third orthogonal moieties; second, third, and fourth orthogonal moieties; etc.). The second orthogonal moiety may be, for example, a dihydropyridazine according to Formula Ia, Formula Ib, Formula Ic, Formula Id, or Formula Ie; a pyridazine according to Formula IIa or Formula IIb; a triazole according to Formula IIIa, Formula IIIb, Formula IIIc; a hydrazide according to Formula IVa or Formula IVb; an oxime according to Formula Va or Formula Vb; a phosphoryl-substituted amide according to Formula Via or Formula VIb; or a thioether according to Formula VIIa or Formula VIIb.

In general, the second orthogonal moiety is different from the first orthogonal moiety (and, in turn, the third orthogonal moiety is different from the second orthogonal moiety and the first orthogonal moiety, and so on). It will be understood however, that the particles can also contain a plurality of first orthogonal moieties and a plurality of second orthogonal moieties, wherein the structures of the first and second orthogonal moieties are different from one another. In embodiments where a particle contains a first dihydropyridazine orthogonal moiety for example, the second orthogonal moiety may be a triazole, a hydrazide, an oxime, a phosphoryl-substituted amide, or a thioether. A particle may contain a plurality of first dihydropyridazine orthogonal moieties and a plurality or second triazole orthogonal moieties, hydrazide orthogonal moieties, oxime orthogonal moieties, phosphoryl-substituted amide orthogonal moieties, or thioether orthogonal moieties. The second orthogonal moiety may containing linking groups -L^{1a}-L^{1b}- and -L^{2a}-L^{2b}- as described above.

In some embodiments, the second orthogonal moiety comprises a dihydropyridazine, a pyridazine, a triazole, a hydrazide, an oxime, a phosphoryl-substituted amide, or a thioether, provided that the second orthogonal moiety is different from the first orthogonal moiety.

In some embodiments, the particle further comprises a second linking moiety between the second orthogonal moiety and the nanoparticle. In some embodiments, the second linking moiety comprises an oligo(ethylene glycol) or a poly(ethylene glycol).

In some embodiments, the first orthogonal moiety or the second orthogonal moiety is a dihydropyridazine. In some embodiments, the first orthogonal moiety or the second orthogonal moiety is a pyridazine.

### Targeting Agents

In some embodiments, at least one of the first effector element and the second effector element is a targeting agent. Generally, the targeting agents of the present invention can associate with any target of interest, such as a target associated with an organ, tissues, cell, extracellular matrix, or intracellular region. In some embodiments, a target can be associated with a particular disease state, such as a cancerous condition. In some embodiments, the targeting component can be specific to only one target, such as a receptor. Suitable targets can include but are not limited to a nucleic acid, such as a DNA, RNA, or modified derivatives thereof. Suitable targets can also include but are not limited to a protein, such as an extracellular protein, a receptor, a cell surface receptor, a tumor-marker, a transmembrane protein, an enzyme, or an antibody. Suitable targets can include a carbohydrate, such as a monosaccharide, disaccharide, or polysaccharide that can be, for example, present on the surface of a cell.

In some embodiments, the targeting agent may include a target ligand (e.g., an RGD-containing peptide), a small molecule mimic of a target ligand (e.g., a peptide mimetic ligand), or an antibody or antibody fragment specific for a particular target. In some embodiments, a targeting agent may include folic acid derivatives, B-12 derivatives, integrin RGD peptides, NGR derivatives, somatostatin derivatives or peptides that bind to the somatostatin receptor, e.g., octreotide and octreotate, and the like. In some embodiments, the targeting agents of the present invention may include an aptamer. Aptamers can be designed to associate with or bind to a target of interest. Aptamers can be comprised of, for example, DNA, RNA, and/or peptides, and certain aspects of aptamers are well known in the art. (See. e.g., Klussman, S., Ed., The Aptamer Handbook, Wiley-VCH (2006); Nissenbaum, E. T., Trends in Biotech. 26(8): 442-449 (2008)).

In some embodiments, the targeting agent is an antibody or antibody fragment. An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively. Light chains are classified as either kappa or lambda. Heavy chains are classified as gamma, mu, alpha, delta, or epsilon, which in turn define the immunoglobulin classes, IgG, IgM, IgA, IgD and IgE, respectively. IgG antibodies are large molecules of about 150 kDa composed of four peptide chains. IgG antibodies contain two identical class y heavy chains of about 50 kDa and two identical light chains of about 25 kDa, thus a tetrameric quaternary structure. The two heavy chains are linked to each other and to a light chain each by disulfide bonds. The resulting tetramer has two identical halves, which together form the Y-like shape. Each end of the fork contains an identical antigen binding site. There are four IgG subclasses (IgG1, 2, 3, and 4) in humans, named in order of their abundance in serum (IgG1 being the most abundant). Typically, the antigen-binding region of an antibody will be most critical in specificity and affinity of binding.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see, Fundamental Immunology (Paul ed., 7th ed. 2012). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990))

The term antibody is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity. “Antibody fragment,” and all grammatical variants thereof, as used herein are defined as a portion of an intact antibody comprising the antigen binding site or variable region of the intact antibody, wherein the portion is free of the constant heavy chain domains (i.e., CH2, CH3, and CH4, depending on antibody isotype) of the Fc region of the intact antibody. Examples of antibody fragments include Fab, Fab′, Fab′-SH, F(ab′)₂, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv (scFv) molecules; (2) single chain polypeptides containing only one light chain variable domain, or a fragment thereof that contains the three CDRs of the light chain variable domain, without an associated heavy chain moiety; (3) single chain polypeptides containing only one heavy chain variable region, or a fragment thereof containing the three CDRs of the heavy chain variable region, without an associated light chain moiety; (4) nanobodies comprising single Ig domains from non-human species or other specific single-domain binding modules; and (5) multispecific or multivalent structures formed from antibody fragments. In an antibody fragment comprising one or more heavy chains, the heavy chain(s) can contain any constant domain sequence (e.g., CH1 in the IgG isotype) found in a non-Fc region of an intact antibody, and/or can contain any hinge region sequence found in an intact antibody, and/or can contain a leucine zipper sequence fused to or situated in the hinge region sequence or the constant domain sequence of the heavy chain(s).

In some embodiments, the antibody is a monoclonal antibody, e.g., abagovomab, abatacept (also known as ORENCIA™), abciximab (also known as REOPRO™, c7E3 Fab), adalimumab (also known as HUMIRA™), adecatumumab, alemtuzumab (also known as CAMPATH™, MabCampath or Campath-1H), altumomab, afelimomab, anatumomab mafenatox, anetumumab, anrukizumab, apolizumab, arcitumomab, aselizumab, atlizumab, atorolimumab, bapineuzumab, basiliximab (also known as SIMULECT™), bavituximab, bectumomab (also known as LYMPHOSCAN™), belimumab (also known as LYMPHO-STAT-B™), bertilimumab, besilesomab, bevacizumab (also known as AVASTIN™), biciromab brallobarbital, bivatuzumab mertansine, campath, canakinumab (also known as ACZ885), cantuzumab mertansine, capromab (also known as PROSTASCINT™), catumaxomab (also known as REMOVAB™), cedelizumab (also known as CIMZIA™), certolizumab pegol, cetuximab (also known as ERBITUX™), clenoliximab, dacetuzumab, dacliximab, daclizumab (also known as ZENAPAX™), denosumab (also known as AMG 162), detumomab, dorlimomab aritox, dorlixizumab, duntumumab, durimulumab, durmulumab, ecromeximab, eculizumab (also known as SOLIRIS™), edobacomab, edrecolomab (also known as Mab17-1A, PANOREX™), efalizumab (also known as RAPTIVA™), efungumab (also known as MYCOGRAB™) elsilimomab, enlimomab pegol, epitumomab cituxetan, efalizumab, epitumomab, epratuzumab, erlizumab, ertumaxomab (also known as REXOMUN™), etanercept (also known as ENBREL™), etaracizumab (also known as etaratuzumab, VITAXIN™, ABEGRIN™) exbivirumab, fanolesomab (also known as NEUTROSPEC™), faralimomab, felvizumab, fontolizumab (also known as HUZAF™), galiximab, gantenerumab, gavilimomab (also known as ABXCBL™), gemtuzumab ozogamicin (also known as MYLOTARG™), golimumab (also known as CNTO 148), gomiliximab, ibalizumab (also known as TNX-355), ibritumomab tiuxetan (also known as ZEVALIN™), igovomab, imciromab, infliximab (also known as REMICADE™), inolimomab, inotuzumab ozogamicin, ipilimumab (also known as MDX-010, MDX-101), iratumumab, keliximab, labetuzumab, lemalesomab, lebrilizumab, lerdelimumab, lexatumumab (also known as, HGS-ETR2, ETR2-ST01), lexitumumab, libivirumab, lintuzumab, lucatumumab, lumiliximab, mapatumumab (also known as HGSETR1, TRM-1), maslimomab, matuzumab (also known as EMD72000), mepolizumab (also known as BOSATRIA™), metelimumab, milatuzumab, minretumomab, mitumomab, morolimumab, motavizumab (also known as NUMAX™), muromonab (also known as OKT3), nacolomab tafenatox, naptumomab estafenatox, natalizumab (also known as TYSABRI™, ANTEGREN™), nebacumab, nerelimomab, nimotuzumab (also known as THERACIM hR3™, THERA-CIM-hR3™, THERALOC™), nofetumomab merpentan (also known as VERLUMA™), ocrelizumab, odulimomab, ofatumumab, omalizumab (also known as XOLAIR™), oregovomab (also known as OVAREX™), otelixizumab, pagibaximab, palivizumab (also known as SYNAGIS™), panitumumab (also known as ABX-EGF, VECTIBIX™), pascolizumab, pemtumomab (also known as THERAGYN™), pertuzumab (also known as 2C4, OMNITARG™), pexelizumab, pintumomab, priliximab, pritumumab, ranibizumab (also known as LUCENTIS™), raxibacumab, regavirumab, reslizumab, rituximab (also known as RITUXAN™, MabTHERA™), rovelizumab, ruplizumab, satumomab, sevirumab, sibrotuzumab, siplizumab (also known as MEDI-507), sontuzumab, stamulumab (also known as MYO-029), sulesomab (also known as LEUKOSCAN™), tacatuzumab tetraxetan, tadocizumab, talizumab, taplitumomab paptox, tefibazumab (also known as AUREXIS™), telimomab aritox, teneliximab, teplizumab, ticilimumab, tocilizumab (also known as ACTEMRA™), toralizumab, tositumomab, trastuzumab (also known as HERCEPTIN™), tremelimumab (also known as CP-675,206), tucotuzumab celmoleukin, tuvirumab, urtoxazumab, ustekinumab (also known as CNTO 1275), vapaliximab, veltuzumab, vepalimomab, visilizumab (also known as NUVION™), volociximab (also known as M200), votumumab (also known as HUMASPECT™), zalutumumab, zanolimumab (also known as HuMAX-CD4), ziralimumab, zolimomab aritox, or the like.

In some embodiments, the antibody or antibody fragment is specific for endothelial growth factor receptor (EGFR); such antibodies include, but are not limited to, cetuximab, panitumumab, necitumumab, and the like. In some embodiments, the antibody or antibody fragment is specific for human epidermal growth factor receptor (HER2); such antibodies include, but are not limited to, trastuzumab, pertuzumab, and the like.

In some embodiments, particles are provided as described above wherein the first effector element and the second effector element are independently selected from the group consisting of an oligopeptide, a polypeptide, an oligonucleotide, and a polynucleotide. In some embodiments, the first effector element and the second effector element are independently selected from the group consisting of an antibody and an antibody fragment.

One of skill in the art will appreciate, however, that particle accumulation at a target site may be due to the enhanced permeability and retention characteristics of certain tissues such as cancer tissues. Accumulation in such a manner often results in part because of particle size and may not require special targeting functionality.

### Therapeutic Agents

In some embodiments, at least one of the first effector element and the second effector element is a therapeutic agent. In general, any therapeutic agent known in the art can be used, including without limitation agents listed in the United States Pharmacopeia (U.S.P.), Goodman and Gilman's The Pharmacological Basis of Therapeutics, 12^{th} Ed., McGraw Hill, 2011; Katzung, Ed., Basic and Clinical Pharmacology, McGraw-Hill/Appleton & Lange, 12^{th} ed., 2012; Physician's Desk Reference, 70^{th} Ed., PDR Network, 2016; and/or The Merck Manual of Diagnosis and Therapy, 19^{th} ed., Merck Publishing Group, 2011; or, in the case of animals, The Merck Veterinary Manual, 10^{th} ed., Merck Publishing Group, 2010; all of which are incorporated herein by reference.

Therapeutic agents can be selected depending on the type of disease desired to be treated. For example, certain types of cancers or tumors, such as carcinoma, sarcoma, leukemia, lymphoma, myeloma, and central nervous system cancers as well as solid tumors and mixed tumors, can involve administration of the same or possibly different therapeutic agents. In certain embodiments, a therapeutic agent can be delivered to treat or affect a cancerous condition in a subject and can include an anticancer agent or cytotoxic agent. Examples of anti-cancer agents include, but are not limited to, alkylating agents (e.g., cyclophosphamide, ifosamide, melphalan, chlorambucil, aziridines, epoxides, alkyl sulfonates), cisplatin and its analogues (e.g., carboplatin, oxaliplatin), antimetabolitites (e.g., methotrexate, 5-fluorouracil, capecitabine, cytarabine, gemcitabine, fludarabine), toposiomerase interactive agents (e.g., camptothecin, irinotecan, topotecan, etoposide, teniposide, doxorubicin, daunorubicin), antimicrotubule agents (e.g., vinca alkaloids, such as vincristine, vinblastine, and vinorelbine; taxanes, such as paclitaxel and docetaxel), interferons, inteleukin-2, histone deacetylase inhibitors, monoclonal antibodies, estrogen modulators (e.g., tamoxifen, toremifene, raloxifene), megestrol, aromatase inhibitors (e.g., letrozole, anastrozole, exemestane, octreotide), octreotide, and anti-androgens (e.g., flutamide, casodex).

In some embodiments, the therapeutic agent may be a corticosteroid. Examples of corticosteroids include, but are not limited to, alclometasone, amcinonide, beclomethasone, betamethasone, clobetasol, clocortolone, cortisol, prednisolone, and pharmaceutically acceptable salts, solvates, clathrates, prodrugs, and active metabolites and stereoisomers thereof. Non-steroidal anti-inflammatory drugs (NSAIDs) may also be included in the liposomes. Examples of NSAIDS include, but are not limited to, acetominophen, apazone, diclofenac, etodolac, fenoprofen, flurbiprofen, ibuprofen, indomethacin, ketorolac, ketoprofen, meclofenamate, mefenamic acid, meloxicam, nabumetone, naproxen, nimesulide, oxaprozin, oxyphenthatrazone, phenylbutazone, piroxicam, salicylates, sulindac, tenoxicam, and tolmetin. Examples of salicylates include, but are not limited to, aspirin, sodium salicylate, choline magnesium trisalicylate, salsalate, diflunisal, salicylsalicylic acid, sulfasalazine, and olsalazine.

In some embodiments, the therapeutic agent may be an antihistamine. Examples of antihistamines include, but are not limited to, acrivastine, astemizole, brompheniramine, carbinoxamine, cetirazine, chlorcyclizine, chlorpheniramine, clemastine, cyclizine, descarboxyloratadine, dimenhydrinate, diphenhydramine, hydroxyzine, levocabastine, loratadine, promethazine, pyrilamine, terfenadine, and ripelennamine.

In some embodiments, the therapeutic agent may be an analgesic. Examples of analgesics include, but are not limited to, bremazocine, buprenorphine, butorphanol, codeine, diprenorphine, dynorphin A, dynorphin B, β-endorphin, ethylketocyclazocine, etorphine, fentanyl, β-funaltrexamine, heroin, hydrocodone, hydromorphone, leu-enkephalin, levophanol, levallorphan, meptazinol, met-enkephalin, methadone, morphine, oxycodone, oxymorphone, nalbuphine, nalmefene, nalorphine, naloxonazine, naloxone, naloxone benzoylhydrazone, naltrexone, naltrindole, α-neoendorphin, nor-binaltorphimine, pentazocine, propoxyphene, and spiradoline.

While a therapeutic agent may be covalently bonded to the surface of a nanoparticle (i.e., as a first effector agent or a second effector agent), therapeutic agents may also be encapsulated by the nanoparticle or otherwise associated with the nanoparticle. In the case of liposome nanoparticles, for example, therapeutic agents may also be non-covalently embedded in the lipid membrane of the liposome or encapsulated in the aqueous core of the liposome. Encapsulation of therapeutic agents can be carried out through a variety of ways known in the art, as disclosed for example in the following references: de Villiers, M. M. et al., Eds., Nanotechnology in Drug Delivery, Springer (2009); Gregoriadis, G., Ed., Liposome Technology: Entrapment of drugs and other materials into liposomes, CRC Press (2006). Loading of liposomes may be carried out, for example, in an active or passive manner. For example, a therapeutic agent can be included during the self-assembly process of the liposomes. In certain embodiments, the therapeutic agent may also be embedded in the liposome bilayer or within multiple layers of a multilamellar liposome. In alternative embodiments, the therapeutic agent can be actively loaded into liposomes. For example, the liposomes can be exposed to conditions, such as electroporation, in which the bilayer membrane is made permeable to a solution containing therapeutic agent thereby allowing for the therapeutic agent to enter into the internal volume of the liposomes.

### Diagnostic Agents

In some embodiments, at least one of the first effector element and the second effector element is a diagnostic agent. The effector element may be any diagnostic agent known in the art, as provided, for example, in the following references: Diagnostic Imaging, 7^{th} Ed Wiley-Blackwell, 2013; Handbook of Targeted Delivery of Imaging Agents, CRC Press, 1995; and Molecular Imaging: Radiopharmaceuticals for PET and SPECT, Springer (2009). A diagnostic agent can be detected by a variety of ways, including as an agent providing and/or enhancing a detectable signal that includes, but is not limited to, gamma-emitting, radioactive, echogenic, optical, fluorescent, absorptive, magnetic or tomography signals. Techniques for imaging the diagnostic agent can include, but are not limited to, single photon emission computed tomography (SPECT), magnetic resonance imaging (MRI), optical imaging, positron emission tomography (PET), computed tomography (CT), x-ray imaging, gamma ray imaging, and the like.

The diagnostic agent may include chelators that bind to metal ions to be used for a variety of diagnostic imaging techniques. Exemplary chelators include but are not limited to ethylenediaminetetraacetic acid (EDTA), [4-(1,4,8, 11-tetraazacyclotetradec-1-yl) methyl]benzoic acid (CPTA), cyclohexanediaminetetraacetic acid (CDTA), ethylenebis(oxyethylenenitrilo)tetraacetic acid (EGTA), diethylenetriaminepentaacetic acid (DTPA), citric acid, hydroxyethyl ethylenediamine triacetic acid (HEDTA), iminodiacetic acid (IDA), triethylene tetraamine hexaacetic acid (TTHA), 1,4,7, 10-tetraazacyclododecane-1,4,7,10-tetra(methylene phosphonic acid) (DOTP), 1,4,8,11-tetraazacyclododecane-1,4,8,11-tetraacetic acid (TETA), 1,4,7,10-tetraazacyclododecane-1,4,7,10-tetraacetic acid (DOTA), and derivatives thereof.

A radioisotope may be incorporated into the liposomes for diagnostic applications as well as therapeutic applications. Radioisotopes include radionuclides that emit gamma rays, positrons, beta and alpha particles, and X-rays. Suitable radionuclides include but are not limited to ²²⁵Ac, ⁷²As, ²¹¹At, ¹¹B, ¹²⁸Ba, ²¹²Bi, ⁷⁵Br, ⁷⁷Br, ¹⁴C, ¹⁰⁹Cd, ⁶²Cu, ⁶⁴Cu, ⁶⁷Cu, ¹⁸F, ⁶⁷Ga, ⁶⁸Ga, ³H, ¹²³I, ¹²⁵I, ¹³⁰I, ¹³¹I, ¹¹¹In, ¹⁷⁷Lu, ¹³N, ¹⁵O, ³²P, ³³P, ²¹²Pb, ¹⁰³Pd, ¹⁸⁶Re, ¹⁸⁸Re, ⁴⁷Sc, ¹⁵³Sm, ⁸⁹Sr, ^{99m}Tc, ⁸⁸Y and ⁹⁰Y. In certain embodiments, radioactive agents can include ¹¹¹In-DTPA, ^{99m}Tc(CO)₃-DTPA, ^{99m}Tc(CO)₃-ENPy2, ^{62/64/67}Cu-TETA, ^{99m}Tc(CO)₃-IDA, and ^{99m}Tc(CO)₃ triamines (cyclic or linear). In other embodiments, the agents can include DOTA and its various analogs with ¹¹¹In, ¹⁷⁷Lu, ¹⁵³Sm, ^{88.90}Y, ^{62/64/67}Cu, or ^{67/68}Ga. In some embodiments, the liposomes can be radiolabeled, for example, by incorporation of lipids attached to chelates, such as DTPA-lipid, as provided in the following references: Phillips et al., Wiley Interdisciplinary Reviews: Nanomedicine and Nanobiotechnology, 1(1): 69-83 (2008); Torchilin, V. P. & Weissig, V., Eds. Liposomes 2nd Ed.: Oxford Univ. Press (2003); Elbayoumi, T. A. & Torchilin, V. P., Eur. J. Nucl. Med. Mol. Imaging 33:1196-1205 (2006); Mougin-Degraef, M. et al., Int'l J. Pharmaceutics 344:110-117 (2007).

In some embodiments, the diagnostic agents may include optical agents such as fluorescent agents, phosphorescent agents, chemiluminescent agents, and the like. Numerous agents (e.g., dyes, probes, labels, or indicators) are known in the art and can be used. (See, e.g., Invitrogen, The Handbook—A Guide to Fluorescent Probes and Labeling Technologies, Tenth Edition (2005)). Fluorescent agents can include a variety of organic and/or inorganic small molecules or a variety of fluorescent proteins and derivatives thereof. For example, fluorescent agents can include but are not limited to cyanines, phthalocyanines, porphyrins, indocyanines, rhodamines, phenoxazines, phenylxanthenes, phenothiazines, phenoselenazines, fluoresceins, benzoporphyrins, squaraines, dipyrrolo pyrimidones, tetracenes, quinolines, pyrazines, corrins, croconiums, acridones, phenanthridines, rhodamines, acridines, anthraquinones, chalcogenopyrylium analogues, chlorins, naphthalocyanines, methine dyes, indolenium dyes, azo compounds, azulenes, azaazulenes, triphenyl methane dyes, indoles, benzoindoles, indocarbocyanines, benzoindocarbocyanines, and BODIPY™ derivatives having the general structure of 4,4-difluoro-4-bora-3a,4a-diaza-s-indacene, and/or conjugates and/or derivatives of any of these.

One of skill in the art will appreciate that particular optical agents used can depend on the wavelength used for excitation, depth underneath skin tissue, and other factors generally well known in the art. For example, optimal absorption or excitation maxima for the optical agents can vary depending on the agent employed, but in general, the optical agents will absorb or be excited by light in the ultraviolet (UV), visible, or infrared (IR) range of the electromagnetic spectrum. For imaging, dyes that absorb and emit in the near-IR (-700-900 nm, e.g., indocyanines) are preferred. For topical visualization using an endoscopic method, any dyes absorbing in the visible range are suitable.

In some embodiments, the diagnostic agents can include but are not limited to magnetic resonance (MR) and x-ray contrast agents that are generally well known in the art, including, for example, iodine-based x-ray contrast agents, superparamagnetic iron oxide (SPIO), complexes of gadolinium or manganese, and the like. (See, e.g., Armstrong et al., Diagnostic Imaging, 5^{th} Ed., Blackwell Publishing (2004)). In some embodiments, a diagnostic agent can include a magnetic resonance (MR) imaging agent. Exemplary magnetic resonance agents include but are not limited to paramagnetic agents, superparamagnetic agents, and the like. Exemplary paramagnetic agents can include but are not limited to gadopentetic acid, gadoteric acid, gadodiamide, gadolinium, gadoteridol , mangafodipir, gadoversetamide, ferric ammonium citrate, gadobenic acid, gadobutrol, or gadoxetic acid. Superparamagnetic agents can include but are not limited to superparamagnetic iron oxide and ferristene. In certain embodiments, the diagnostic agents can include x-ray contrast agents. Examples of x-ray contrast agents include, without limitation, iopamidol, iomeprol, iohexol, iopentol, iopromide, iosimide, ioversol, iotrolan, iotasul, iodixanol, iodecimol, ioglucamide, ioglunide, iogulamide, iosarcol, ioxilan, iopamiron, metrizamide, iobitridol and iosimenol. In certain embodiments, the x-ray contrast agents can include iopamidol, iomeprol, iopromide, iohexol, iopentol, ioversol, iobitridol, iodixanol, iotrolan and iosimenol.

As for the therapeutic agents described above, the diagnostic agents can also be encapsulated by a nanoparticle or otherwise associated with the nanoparticle (e.g., embedded or encapsulated in a liposome). Similarly, loading of the diagnostic agents can be carried out through a variety of ways known in the art, as disclosed for example in the following references: de Villiers, M. M. et al., Eds., Nanotechnology in Drug Delivery, Springer (2009); Gregoriadis, G., Ed., Liposome Technology: Entrapment of drugs and other materials into liposomes, CRC Press (2006).

### Liposomes and Other Nanoparticles

A number of nanoparticles can be orthogonally functionalized using the methods disclosed herein; such nanoparticles include, but are not limited to, polymer nanoparticles (e.g., biodegradable particles such as poly(lactic-co-glycolic acid) (PLGA) nanoparticles) and inorganic nanoparticles (e.g., semiconducting nanoparticles such as cadmium selenide nanoparticles or noble metal nanoparticles such as gold nanoparticles).

In certain embodiments, the nanoparticle is a liposome comprising a lipid membrane. The liposomes can contain a variety of lipids, including fats, waxes, steroids, sterols, cholesterol, fat-soluble vitamins, monoglycerides, diglycerides, phospholipids, sphingolipids, glycolipids, cationic or anionic lipids, derivatized lipids, and the like, as described in more detail below. In some such embodiments, the orthogonal moieties contain lipid groups which are embedded in the lipid membrane (e.g., a phospholipid membrane of a liposome such as a small unilamellar vesicle).

The liposomes may be unilamellar vesicles which are comprised of a single lipid bilayer and generally have a diameter in the range of about 20 to about 400 nm. Liposomes can also be multilamellar, which generally have a diameter in the range of 1 to 10 μm. In some embodiments, liposomes can include multilamellar vesicles (MLVs; from about 1 μm to about 10 μm in size), large unilamellar vesicles (LUVs; from a few hundred nanometers to about 10 μm in size), and small unilamellar vesicles (SUVs; from about 20 nm to about 200 nm in size). In some instances, the liposomes are small unilamellar vesicles.

The liposomes may contain reactive phospholipids including, e.g., phosphatidyl-ethanolamines (PEs), phosphatidylglycerols (PGs), and phosphatidylserines (PSs). Reactive phospholipid headgroups may be chemically modified with linking moieties and/or reactive groups suitable for connecting the lipid groups to orthogonal moieties. Examples reactive phospholipids include, but are not limited to, dimyristoylphosphatidylglycerol (DMPG), distearoylphosphatidylglycerol (DSPG), dioleoylphosphatidylglycerol (DOPG), dipalmitoylphosphatidylglycerol (DPPG), dimyristoylphosphatidylserine (DMPS), distearoylphosphatidylserine (DSPS), dioleoylphosphatidylserine (DOPS), dipalmitoylphosphatidylserine (DPPS), dioleoylphosphatidylethanolamine (DOPE), palmitoyloleoylphosphatidylethanolamine (POPE), dipalmitoylphosphatidylethanolamine (DPPE), dimyristoylphosphoethanolamine (DMPE), distearoylphosphatidylethanolamine (DSPE), 16-O-monomethyl PE, 16-O-dimethyl PE, 18-1-trans PE, 1-stearoyl-2-oleoyl-phosphatidyethanolamine (SOPE), and dielaidoylphosphoethanolamine (transDOPE). Phospholipids may further include reactive functional groups for further derivatization. Examples of such reactive lipids include, but are not limited to, dioleoylphosphatidylethanolamine-4-(N-maleimidomethyl)-cyclohexane-1-carboxylate (DOPE-mal) and dipalmitoylphosphatidylethanolamine-N-succinyl (succinyl-PE).

Accordingly, some embodiments provide nanoparticles wherein the first orthogonal moiety further comprises a first lipid group and the second orthogonal moiety further comprises a second lipid group, and wherein the first lipid group and the second lipid group are embedded in the lipid membrane. In some embodiments, the lipid membrane is a phospholipid membrane, the first lipid group is a phospholipid, and the second lipid group is a phospholipid. In some embodiments, the first lipid group is a phosphatidylethanolamine and the second lipid group is a phosphatidylethanolamine.

In some embodiments, the first orthogonal moiety or the second orthogonal moiety is a dihydropyridazine according to Formula If

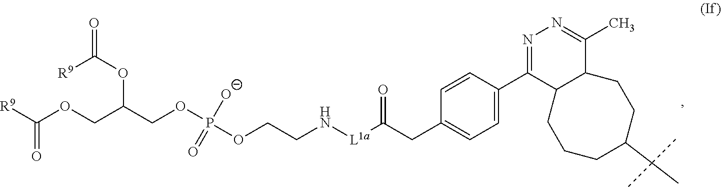

wherein L^{1a} is as described above, each R⁹ is independently selected from C₉-C₂₁ alkyl and C₉-C₂₁ alkenyl. In some embodiments, L^{1a} is a divalent polymer moiety. In some embodiments, each R⁹ is independently selected from C₁₃-C₁₇ alkyl and C₁₃-C₁₇ alkenyl. In some embodiments, L^{1a} is a divalent polymer moiety and each R⁹ is independently selected from C₁₃-C₁₇ alkyl and C₁₃-C₁₇ alkenyl. In some such embodiments, the R⁹ groups are embedded in the lipid membrane of a liposome.

In some embodiments, the first orthogonal moiety or the second orthogonal moiety is a dihydropyridazine according to Formula Ig

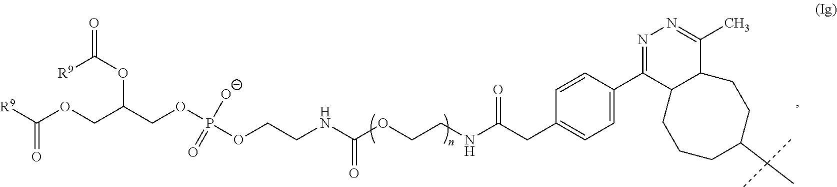

wherein subscript n is an integer ranging from about 15 to about to about 200, and each R⁹ is independently selected from C₉-C₂₁ alkyl and C₉-C₂₁ alkenyl. In some embodiments, subscript n ranges from about 30 to about 90. In some such embodiments, the R⁹ groups are embedded in the lipid membrane of a liposome.

In some embodiments, the first orthogonal moiety or the second orthogonal moiety is a triazole according to Formula IIIe

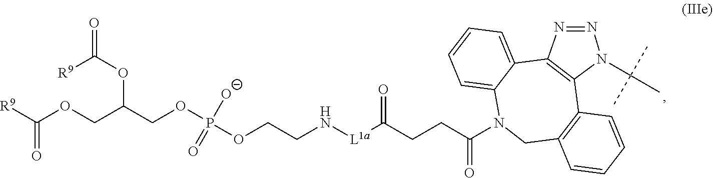

wherein L^{1a} is as described above, each R⁹ is independently selected from C₉-C₂₁ alkyl and C₉-C₂₁ alkenyl. In some embodiments, L^{1a} is a divalent polymer moiety. In some embodiments, each R⁹ is independently selected from C₁₃-C₁₇ alkyl and C₁₃-C₁₇ alkenyl. In some embodiments, L^{1a} is a divalent polymer moiety and each R⁹ is independently selected from C₁₃-C₁₇ alkyl and C₁₃-C₁₇ alkenyl. In some such embodiments, the R⁹ groups are embedded in the lipid membrane of a liposome.

In some embodiments, the first orthogonal moiety or the second orthogonal moiety is a triazole according to Formula IIIf

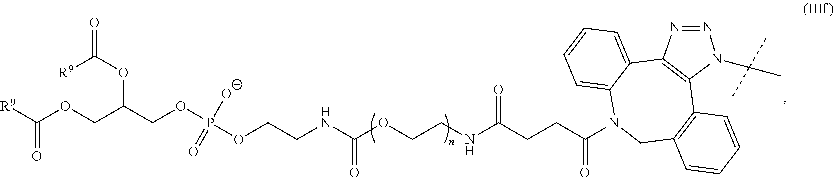

wherein subscript n is an integer ranging from about 15 to about to about 200, and each R⁹ is independently selected from C₉-C₂₁ alkyl and C₉-C₂₁ alkenyl. In some embodiments, subscript n ranges from about 30 to about 90. In some such embodiments, the R⁹ groups are embedded in the lipid membrane of a liposome.

The liposomes can also containing non-reactive lipids. In some embodiments, each of the non-reactive lipids is independently selected from the group consisting of a phosphatidylcholine, a PEGylated phosphatidylethanolamine, and a sterol.

Suitable phosphatidylcholine lipids include saturated PCs and unsaturated PCs. Examples of saturated PCs include 1,2-dilauroyl-sn-glycero-3-phosphocholine (DLPC), 1,2-dimyristoyl-sn-glycero-3-phosphocholine (dimyristoylphosphatidylcholine; DMPC), 1,2-distearoyl-sn-glycero-3-phosphocholine (distearoylphosphatidylcholine; DSPC), 1,2-dioleoyl-sn-glycero-3-phosphocholine (DOPC), 1,2-dipalmitoyl-sn-glycero-3-phosphocholine (dipalmitoyl-phosphatidylcholine; DPPC), 1-myristoyl-2-palmitoyl-sn-glycero-3-phosphocholine (MPPC), 1-palmitoyl-2-myristoyl-sn-glycero-3-phosphocholine (PMPC), 1-myristoyl-2-stearoyl-sn-glycero-3-phosphocholine (MSPC), 1-palmitoyl-2-stearoyl-sn-glycero-3-phosphocholine (PSPC), 1-stearoyl-2-palmitoyl-sn-glycero-3-phosphocholine (SPPC), and 1-stearoyl-2-myristoyl-sn-glycero-3-phosphocholine (SMPC).

Examples of unsaturated PCs include, but are not limited to, 1,2-dimyristoleoyl-sn-glycero-3-phosphocholine, 1,2-dimyristelaidoyl-sn-glycero-3-phosphocholine, 1,2-dipalmito-leoyl-sn-glycero-3-phosphocholine, 1,2-dipalmitelaidoyl-sn-glycero-3-phosphocholine, 1,2-dioleoyl-sn-glycero-3-phosphocholine (DOPC), 1,2-dielaidoyl-sn-glycero-3-phosphocholine, 1,2-dipetroselenoyl-sn-glycero-3-phosphocholine, 1,2-dilinoleoyl-sn-glycero-3-phosphocholine, 1-palmitoyl-2-oleoyl-sn-glycero-3-phosphocholine (palmitoyloleoylphosphatidylcholine; POPC), 1-palmitoyl-2-linoleoyl-sn-glycero-3-phosphocholine, 1-stearoyl-2-oleoyl-sn-glycero-3-phosphocholine (SOPC), 1-stearoyl-2-linoleoyl-sn-glycero-3-phosphocholine, 1-oleoyl-2-myrist-oyl-sn-glycero-3-phosphocholine (OMPC), 1-oleoyl-2-palmitoyl-sn-glycero-3-phosphocholine (OPPC), and 1-oleoyl-2-stearoyl-sn-glycero-3-phosphocholine (OSPC). Lipid extracts, such as egg PC, heart extract, brain extract, liver extract, soy PC, and hydrogenated soy PC (HSPC) are also useful. Other phospholipids including, but are not limited to, phosphatidic acids (PAs), phosphatidylserines (PSs), and phosphatidylinositols (PIs) may also be used in the lipid compositions.

In some embodiments, the phosphatidylcholine lipid is selected from the group consisting of DPPE, DPPC, DSPC, HSPC, and mixtures thereof. The liposomes can contain any suitable amount of phosphatidylcholine or phosphatidylcholine mixture. For example, the amount of phosphatidylcholine or phosphatidylcholine mixture in the liposomes can range from about 40 mol % to about 43 mol %, or from about 43 mol % to about 46 mol %, or from about 46 mol % to about 49 mol %, or from about 49 mol % to about 52 mol %, or from about 52 mol % to about 55 mol %, or from about 55 mol % to about 58 mol %, or from about 58 mol % to about 61 mol %, or from about 61 mol % to about 64 mol %, or from about 64 mol % to about 67 mol %, or from about 67 mol % to about 70 mol %. The amount of phosphatidylcholine or phosphatidylcholine mixture in the liposomes can range from about 40 mol % to about 70 mol %, or from about 42 mol % to about 68 mol %, or from about 44 mol % to about 66 mol %, or from about 46 mol % to about 64 mol %, or from about 48 mol % to about 62 mol %, or from about 50 mol % to about 60 mol %, or from about 52 mol % to about 58 mol %, or from about 54 mol % to about 56 mol %. In some embodiments, the liposomes contain 50-65 mol % of a phosphatidylcholine lipid or mixture of phosphatidylcholine lipids or 45-70 mol % of a phosphatidylcholine lipid or mixture of phosphatidylcholine lipids. The liposomes can contain, for example, 50, 51, 52, 53, 54, 55, 56, 57, 58, 59, 60, 61, 62, 63, 64 or 65 mol % phosphatidylcholine (e.g., DPPE). In some embodiments, the liposomes contain about 59 mol % phosphatidylcholine (e.g., DPPE).

In some embodiments, the liposomes further contain one or more steroids, characterized by the presence of a fused, tetracyclic gonane ring system. Examples of steroids include, but are not limited to, cholic acid, progesterone, cortisone, aldosterone, testosterone, dehydroepiandrosterone, and sterols such as estradiol and cholesterol. In some cases, the sterol is cholesterol or a cholesterol derivative, such as 2,15-dimethyl-14-(1,5-dimethylhexyl)-tetracyclo[8.7.0.0^{2,7}.0^{11,5}]heptacos-7-en-5-ol) or cholesteryl pelargonate. Other sterols, including stigmasterol, campesterol, zymostenol, sitosterol, and pregnenolone, can also be included in the liposomes. The liposomes can contain any suitable amount of sterol. For example, the amount of the sterol or sterol mixture in the liposomes can range from about 20 mol % to about 24 mol %, or from about 24 mol % to about 28 mol %, or from about 28 mol % to about 32 mol %, or from about 32 mol % to about 36 mol %, or from about 36 mol % to about 40 mol %, or from about 40 mol % to about 44 mol %, or from about 44 mol % to about 48 mol %, or from about 48 mol % to about 50 mol %. The amount of the sterol or sterol mixture in the liposomes can range from about 20 mol % to about 50 mol %, or from about 23 mol % to about 47 mol %, or from about 26 mol % to about 44 mol %, or from about 29 mol % to about 41 mol %, or from about 32 mol % to about 38 mol %, or from about 35 mol % to about 35 mol %, or from about 38 mol % to about 32 mol %, or from about 41 mol % to about 29 mol %, or from about 44 mol % to about 26 mol %, or from about 47 mol % to about 23 mol %, or from about 50 mol % to about 20 mol %. In some embodiments, the liposomes can contain about 20-50 mol % sterol, or about 25-35 mol % sterol. The liposomes can contain, for example, 24, 26, 27, 28, 29, 30, 31, 32, 33, 34, 35, 36, 37, 38, 39, 40, 41, 42, 43, 44, or 45 mol % sterol. In some embodiments, the liposomes contain 30-40 mol % cholesterol. In some embodiments, the liposomes contain about 36 mol % cholesterol.

In some embodiments, the liposomes further contain a (polyethylene glycol)-lipid (i.e., a “PEG-lipid”). The presence of PEG on the surface of a liposome has been shown to extend blood-circulation time while reducing mononuclear phagocyte system uptake, creating so-called “stealth” liposomes as described in U.S. Pat. Nos. 5,013,556 and 5,827,533, each of which is hereby incorporated by reference in its entirety. The liposomes may include any suitable PEG-lipid. In some embodiments, the PEG-lipid is a PEGylated phosphatidylethanolamine (e.g., a diacyl-phosphatidylethanolamine-N-[methoxy(polyethylene glycol)]). The molecular weight of the poly(ethylene glycol) in the PEG-lipid is generally in the range of from about 500 Daltons (Da) to about 5000 Da. The poly(ethylene glycol) can have a molecular weight of, for example, about 750 Da, about 1000 Da, about 2500 Da, or about 5000 Da, or about 10,000 Da, or any molecular weight within this range. In some embodiments, the PEG-lipid is selected from distearoyl-phosphatidylethanolamine-N-[methoxy(polyethylene glycol)-2500] (DSPE-PEG-2500) and distearoyl-phosphatidylethanolamine-N-[methoxy(polyethylene glycol)-5000] (DSPE-PEG-5000). In some embodiments, the PEG-lipid is DSPE-PEG-2500. In some embodiments, the PEG-lipid is DSPE-PEG-5000.

The liposomes may contain any suitable amount of PEG-lipid. For example, the amount of the PEG-lipid in the liposomes can range from about 1 mol % to about 2 mol %, or from about 2 mol % to about 3 mol %, or from about 3 mol % to about 4 mol %, or from about 4 mol % to about 5 mol %, or from about 5 mol % to about 6 mol %, or from about 6 mol % to about 7 mol %, or from about 7 mol % to about 8 mol %, or from about 8 mol % to about 9 mol %, or from about 9 mol % to about 10 mol %. The amount of the PEG-lipid in the liposomes can range from about 1 mol % to about 10 mol %, or from about 2 mol % to about 9 mol %, or from about 3 mol % to about 8 mol %, or from about 4 mol % to about 7 mol %. In some embodiments, the liposomes contain 1-8 mol % of the PEG-lipid. The liposomes can contain, for example, 1, 2, 3, 4, 5, 6, 7, or 8 mol % PEG-lipid. In some embodiments, the liposomes contain 2-6 mol % PEG-lipid (e.g., DSPE-PEG-2000 or DSPE-PEG-5000). In some embodiments, the liposomes contain about 5 mol % PEG-lipid (e.g., DSPE-PEG-2000 or DSPE-PEG-5000).

The liposomes may contain any suitable amount of a lipid containing an effector element, orthogonal moiety, or reactive functional group. For example, the amount of the lipid containing the effector element, the orthogonal moiety, or the reactive functional group can range from about 0.01 mol % to about 0.1 mol %, or from about 0.1 mol % to about 1 mol %, or from about 1 mol % to about 2 mol %, or from about 2 mol % to about 3 mol %, or from about 3 mol % to about 4 mol %, or from about 4 mol % to about 5 mol %, or from about 5 mol % to about 6 mol %, or from about 6 mol % to about 7 mol %, or from about 7 mol % to about 8 mol %, or from about 8 mol % to about 9 mol %, or from about 9 mol % to about 10 mol %. The amount of the lipid containing the effector element, the orthogonal moiety, or the reactive functional group in the liposomes can range from about 0.01 mol % to about 10 mol %, or from about 0.1 mol % to about 6 mol %, or from about 0.1 mol % to about 4 mol %, or from about 0.1 mol % to about 2 mol %. In some embodiments, the liposomes contain 0.1-1 mol % of the lipid containing the effector element, the orthogonal moiety, or the reactive functional group. The liposomes can contain, for example, 0.1, 0.2, 0.3, 0.4, 0.5, 0.6, 0.7, 0.8, or 0.9 mol % of the lipid containing the effector element, the orthogonal moiety, or the reactive functional group. In some embodiments, the liposomes contain 0.2-0.8 mol % of the lipid containing the effector element, the orthogonal moiety, or the reactive functional group. In some embodiments, the liposomes contain about 0.6 mol % of the lipid containing the effector element, the orthogonal moiety, or the reactive functional group.

In some embodiments, the liposomes contain from about 50 mol % to about 70 mol % DPPE, from about 30 mol % to about 40 mol % cholesterol, from about 0.1 mol % to about 0.5 mol % DPPE-Cy5.5, and from about 1.5 mol % to about 8 mol % DSPE-PEGSK. In some embodiments, the liposomes contain from about 50 mol % to about 65 mol % HSPC, from about 35 mol % to about 45 mol % cholesterol, from about 0.1 mol % to about 0.5 mol % DPPE-Cy5.5, and from about 1.5 mol % to about 8 mol % DSPE-PEGSK.

In some embodiments, the particle further comprises a therapeutic agent, diagnostic agent, or a combination thereof.

Also provided is a population of particles according to the embodiments described above. Typically, the liposomes will range from tens of nanometers to a few microns in diameter. In some embodiments, the average diameter of the particles is 100 nanometers or less. The diameter of the liposomes can range, for example, from about 5 nm to about 10 nm, or from about 10 nm to about 20 nm, or from about 20 nm to about 30 nm, or from about 30 nm to about 40 nm, or from about 40 nm to about 50 nm, or from about 50 nm to about 60 nm, or from about 60 nm to about 70 nm, or from about 70 nm to about 80 nm, or from about 80 nm to about 90 nm, or from about 90 nm to about 95 nm. The diameter of the liposomes can range from about 35 nm to about 40 nm, or from about 40 nm to about 45 nm, or from about 45 nm to about 50 nm, or from about 40 nm to about 60 nm, or from about 20 nm to about 80 nm, or from about 10 nm to about 90 nm. The diameter of the liposomes can be about 40, 41, 42, 43, 44, 45, 46, 47, 48, 49 50, 51, 52, 53, 54, 55, 60, 65, 70, 75, 80, 85, 90, 95, or 99 nm.

The liposome populations described herein typically have low polydispersities, generally having a polydispersity index that is less than 0.3, less than 0.2, less than 0.15, or less than 0.10, as measured by DLS. In some embodiments, the polydispersity index of the population is 0.20 or less.

### IV. METHODS FOR MAKING MULTIPLEXED PARTICLES

Also provided here is a method of making a particle comprising a first effector element and a second effector element. The method includes:
(i) providing a nanoparticle having a first reactive functional group and a second reactive functional group; and
(ii) combining the nanoparticle with a first reactive effector element and a second reactive effector element under conditions sufficient to form:
   (a) a first orthogonal moiety covalent bonding the first reactive functional group to the first reactive recognition element and
   (b) a second orthogonal moiety covalently bonding the second reactive functional group to the second reactive effector element,
wherein the first orthogonal moiety and the second orthogonal moiety have different chemical structures;
thereby forming the particle.

In some embodiments, the first effector element and the second effector element are independently selected from the group consisting of a targeting agent, a therapeutic agent, and a diagnostic agent.

### Functional Groups for Orthogonal Reactions

In some embodiments, the first reactive functional group is selected from the group consisting of a tetrazine, a cycloalkene, a cycloalkyne, a linear alkyne, an aminooxy compound, a hydrazide, a ketone, an azide, a phosphine, a thiol, and a maleimide.

In some embodiments, the first reactive functional group or the second reactive functional group comprises a tetrazine according to Formula XI

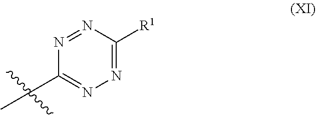

wherein the wavy line represents the point of connection to the particle, and R¹ is selected from the group consisting of H and C₁₋₆ alkyl. Tetrazines according to Formula XI can be bonded to the nanoparticle directly or via linkers as described above. For example, a tetrazine comprising a lipid group can be imbedded in the membrane of a liposome. Tetrazines according to Formula XI can also be bonded to effector elements in a similar fashion, so as to provide reactive effector elements.

In some embodiments, the first reactive functional group or the second reactive functional group comprises a cycloalkane such as a trans-cyclooctene according to Formula XII.

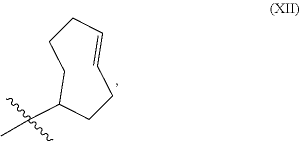 [SMILES: *C1CC/C=C/CCC1]

the wavy line represents the point of connection to the particle. Trans-cyclooctenes according to Formula XII can be bonded to the nanoparticle directly or via linkers as described above. For example, a trans-cyclooctene comprising a lipid group can be imbedded in the membrane of a liposome. Trans-cyclooctenes according to Formula XII can also be bonded to effector elements in a similar fashion, so as to provide reactive effector elements.

In some embodiments, the first reactive functional group or the second reactive functional group comprises a cycloalkyne such as a cyclooctyne according to Formula XIII:

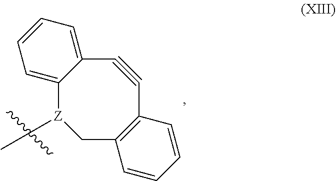

wherein the wavy line represents the point of connection to the particle, and Z is N or CH. Cyclooctynes according to Formula XIII can be bonded to the nanoparticle directly or via linkers as described above. For example, a cyclooctyne comprising a lipid group can be imbedded in the membrane of a liposome. Cyclooctynes according to Formula XIII can also be bonded to effector elements in a similar fashion, so as to provide reactive effector elements.

In some embodiments, the first reactive functional group or the second reactive functional group comprises a linear alkyne such as a linear alkyne according to Formula XIV:

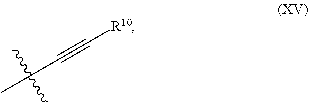

wherein the wavy line represents the point of connection to the particle, and R¹⁰ is selected from the group consisting of H and C₁₋₆ alkyl, C₃₋₈ cycloalkyl. Linear alkynes according to Formula XV can be bonded to the nanoparticle directly or via linkers as described above. For example, a linear alkyne comprising a lipid group can be imbedded in the membrane of a liposome. Linear alkynes according to Formula XV can also be bonded to effector elements in a similar fashion, so as to provide reactive effector elements.

In some embodiments, the first reactive functional group or the second reactive functional group comprises an aminooxy compound having the formula —ONH₂. Aminooxy compounds can be bonded to the nanoparticle directly or via linkers as described above. For example, an aminooxy compound comprising a lipid group can be imbedded in the membrane of a liposome. Aminooxy compounds can also be bonded to effector elements in a similar fashion, so as to provide reactive effector elements.

In some embodiments, the first reactive functional group or the second reactive functional group comprises a hydrazide having the formula —C(O)NHNH₂. Hydrazides can be bonded to the nanoparticle directly or via linkers as described above. For example, a hydrazide comprising a lipid group can be imbedded in the membrane of a liposome. Hydrazides can also be bonded to effector elements in a similar fashion, so as to provide reactive effector elements.

In some embodiments, the first functional group or the second reactive functional group comprises a ketone such as a ketone according to Formula XVI:

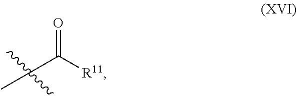

wherein the wavy line represents the point of connection to the particle, and R¹¹ is selected from the group consisting of C₁₋₆ alkyl, C₃₋₈ cycloalkyl, C₆₋₁₀ aryl, 5-to-12-membered heterocyclyl, and 5-to-12-membered heteroaryl. In some embodiments, R¹¹ is C₁₋₆ alkyl. Ketones according to Formula XVI can be bonded to the nanoparticle directly or via linkers as described above. For example, a ketone comprising a lipid group can be imbedded in the membrane of a liposome. Ketones according to Formula XVI can also be bonded to effector elements in a similar fashion, so as to provide reactive effector elements.

In some embodiments, the first functional group or the second reactive functional group comprises an azide having the formula —N₃. Azides can be bonded to the nanoparticle directly or via linkers as described above. For example, an azide comprising a lipid group can be imbedded in the membrane of a liposome. Azides can also be bonded to effector elements in a similar fashion, so as to provide reactive effector elements.

In some embodiments, the first functional group or the second reactive functional group comprises a phosphine such as a phosphine according to Formula XVII:

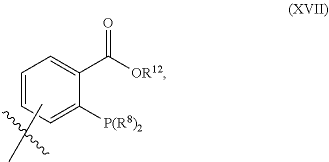

wherein the wavy line represents the point of connection to the particle; each R⁸ is independently selected from the group consisting of C₁₋₆ alkyl, C₃₋₈ cycloalkyl, 5-to-12-membered heterocyclyl, C₆₋₁₀ aryl, and 5-to-12-membered heteroaryl; and R¹² is independently selected from H and C₁₋₆ alkyl. In some embodiments, each R⁸ is phenyl. Phosphines according to Formula XVII can be bonded to the nanoparticle directly or via linkers as described above. For example, a phosphine comprising a lipid group can be imbedded in the membrane of a liposome. Phosphines according to Formula XVII can also be bonded to effector elements in a similar fashion, so as to provide reactive effector elements.

In some embodiments, the first functional group or the second reactive functional group comprises a thiol having the formula —SH. Thiols can be bonded to the nanoparticle directly or via linkers as described above. For example, a thiol comprising a lipid group can be imbedded in the membrane of a liposome. Thiols can also be bonded to effector elements in a similar fashion, so as to provide reactive effector elements.

In some embodiments, the first functional group or the second reactive functional group comprises a maleimide such as a maleimide according to Formula XVIII:

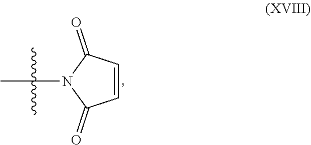 [SMILES: *N1C(=O)C=CC1=O]

wherein the wavy line represents the point of connection to the particle. Maleimides according to Formula XVIII can be bonded to the nanoparticle directly or via linkers as described above. For example, a maleimide comprising a lipid group can be imbedded in the membrane of a liposome. Maleimides according to Formula XVIII can also be bonded to effector elements in a similar fashion, so as to provide reactive effector elements.

In some embodiments, the particle further comprises a first linking moiety between the nanoparticle and the first reactive functional group, as described above. In some embodiments, the first linking moiety comprises an oligo(ethylene glycol) or a poly(ethylene glycol).

In some embodiments, the second reactive functional group is selected from the group consisting of a tetrazine (e.g., a tetrazine according to Formula XI), a cycloalkane (e.g., a trans-cyclooctene according to Formula XII), a cycloalkyne (e.g., a cyclooctyne according to Formula XIII), a linear alkyne (e.g., a linear alkyne according to Formula XIV), an aminooxy compound, a hydrazide, a ketone (e.g., a ketone according to Formula XVI), an azide, a phosphine (e.g., a phosphine according to Formula XVII), a thiol, and a maleimide (e.g., a maleimide according to Formula XVIII), provided that the second reactive functional group is different from the first reactive functional group. In some embodiments, the particle further comprises a second linking moiety between the nanoparticle and the first reactive functional group, as described above. In some embodiments, the second linking moiety comprises an oligo(ethylene glycol) or a poly(ethylene glycol).

Any of the effector elements described above (i.e., targeting agents, therapeutic agents, and/or diagnostic agents) can be used in the methods. In some embodiments, at least one of the effector elements is a targeting agent. In some embodiments, the targeting agent is selected from the group consisting of an oligopeptide, a polypeptide, an oligonucleotide, and a polynucleotide. In some embodiments, the targeting agent is selected from the group consisting of an antibody and an antibody fragment.

Preparation of reactive effector elements

Effector elements such as antibodies or antibody fragments can be modified to include a suitable orthogonal reactive functional groups using various chemistries for bioconjugation, which typically include the use of reagents having reactive linker groups. A wide variety of such reagents are known in the art. Examples of such reagents include, but are not limited to, N-hydroxysuccinimidyl (NHS) esters and N-hydroxysulfosuccinimidyl (sulfo-NHS) esters (amine reactive); carbodiimides (amine and carboxyl reactive); hydroxymethyl phosphines (amine reactive); maleimides (sulfhydryl reactive); aryl azides (primary amine reactive); pentafluorophenyl (PFP) esters (amine reactive); imidoesters (amine reactive); isocyanates (hydroxyl reactive); vinyl sulfones (sulfhydryl, amine, and hydroxyl reactive); and pyridyl disulfides (sulfhydryl reactive). Further reagents include but are not limited to those groups and methods described in Hermanson, Bioconjugate Techniques 2nd Edition, Academic Press, 2008.

Accordingly, a effector element can be converted to a reactive effector element using a reagent selected from:

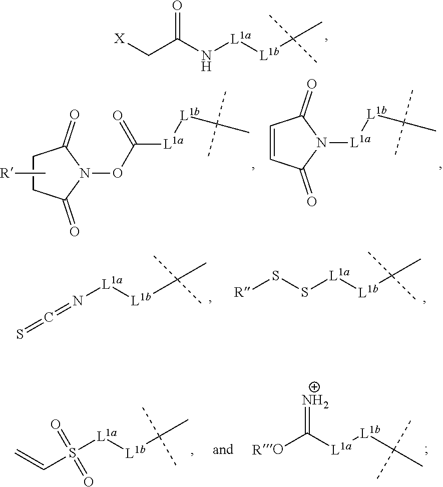

wherein X is halogen (e.g., iodo or chloro); R′ is H or sulfo; R″ is optionally substituted C₆₋₁₀ aryl (e.g., 3-carboxy-4-nitrophenyl) or optionally substituted heteroaryl (e.g., pyridin-2-yl); R′″ is optionally substituted alkyl (e.g., methoxy); L^{1a} and L^{1b} are as described above; and the dashed line represents the point of connection to the orthogonal functional group (e.g., to a tetrazine, a trans-cyclooctene, or another functional group described above).

Reaction mixtures for installing an orthogonal functional group in a reactive effector element can contain additional reagents of the sort typically used in bioconjugation reactions. For example, in certain embodiments, the reaction mixtures can contain buffers (e.g., 2-(N-morpholino)ethanesulfonic acid (MES), 2-[4-(2-hydroxyethyl)piperazin-1-yl]ethanesulfonic acid (HEPES), 3-morpholinopropane-1-sulfonic acid (MOPS), 2-amino-2-hydroxymethyl-propane-1,3-diol (TRIS), potassium phosphate, sodium phosphate, phosphate-buffered saline, sodium citrate, sodium acetate, and sodium borate), cosolvents (e.g., dimethylsulfoxide, dimethylformamide, ethanol, methanol, tetrahydrofuran, acetone, and acetic acid), salts (e.g., NaCl, KCl, CaCl₂, and salts of Mn²⁺ and Mg²⁺), detergents/surfactants (e.g., a non-ionic surfactant such as N,N-bis[3-(D-gluconamido)propyl]cholamide, polyoxyethylene (20) cetyl ether, dimethyldecylphosphine oxide, branched octylphenoxy poly(ethyleneoxy)ethanol, a polyoxyethylene-polyoxypropylene block copolymer, t-octylphenoxypolyethoxyethanol, polyoxyethylene (20) sorbitan monooleate, and the like; an anionic surfactant such as sodium cholate, N-lauroylsarcosine, sodium dodecyl sulfate, and the like; a cationic surfactant such as hexdecyltrimethyl ammonium bromide, trimethyl(tetradecyl) ammonium bromide, and the like; or a zwitterionic surfactant such as an amidosulfobetaine, 3-[(3-cholamidopropyl)dimethyl-ammonio]-1-propanesulfonate, and the like), chelators (e.g., ethylene glycol-bis(2-aminoethylether)-N,N,N′,N′-tetraacetic acid (EGTA), 2-({2-[bis(carboxymethyl)amino]ethyl} (carboxymethyl)amino)acetic acid (EDTA), and 1,2-bis(o-aminophenoxy)ethane-N,N,N′,N′-tetraacetic acid (BAPTA)), and reducing agents (e.g., dithiothreitol (DTT), β-mercaptoethanol (BME), and tris(2-carboxyethyl)phosphine (TCEP)). Buffers, cosolvents, salts, detergents/surfactants, chelators, and reducing agents can be used at any suitable concentration, which can be readily determined by one of skill in the art. In general, buffers, cosolvents, salts, detergents/surfactants, chelators, and reducing agents are included in reaction mixtures at concentrations ranging from about 1 μM to about 1 M. For example, a buffer, a cosolvent, a salt, a detergent/surfactant, a chelator, or a reducing agent can be included in a reaction mixture at a concentration of about 1 μM, or about 10 μM, or about 100 μM, or about 1 mM, or about 10 mM, or about 25 mM, or about 50 mM, or about 100 mM, or about 250 mM, or about 500 mM, or about 1 M.

The reactions are conducted under conditions sufficient to install the orthogonal functional group in the effector element. The reactions can be conducted at any suitable temperature. In general, the reactions are conducted at a temperature of from about 4° C. to about 40° C. The reactions can be conducted, for example, at about 25° C. or about 37° C. The reactions can be conducted at any suitable pH. In general, the reactions are conducted at a pH of from about 4.5 to about 10. The reactions can be conducted, for example, at a pH of from about 5 to about 9. The reactions can be conducted for any suitable length of time. In general, the reaction mixtures are incubated under suitable conditions for anywhere between about 1 minute and several hours. The reactions can be conducted, for example, for about 1 minute, or about 5 minutes, or about 10 minutes, or about 30 minutes, or about 1 hour, or about 2 hours, or about 4 hours, or about 8 hours, or about 12 hours, or about 24 hours, or about 48 hours, or about 72 hours. Other reaction conditions may be employed, depending on the identity of the effector element and the reagent used for installing the orthogonal reactive group.

### Preparation of Liposomes

As described above, the nanoparticle can be a liposome comprising a lipid membrane. In some embodiments, the lipid membrane comprises one or more phospholipids, one or more sterols, one or more PEG-lipids, or a combination thereof. In some such embodiments, the first orthogonal comprises a first lipid group and the second orthogonal moiety comprises a second lipid group, and wherein the first lipid group and the second lipid group are embedded in the lipid membrane.

Liposomes can be prepared according to a number of processes including, but not limited to, extrusion, probe sonication, and bath sonication. Preparation of small unilamellar vesicles can be conducted as described, for example, in U.S. Pat. Nos. 4,752,425; 4,737,323; and 6,623,671, which are incorporated herein by reference in their entirety. As a non-limiting example, a mixture of a phospholipid (e.g., DPPE), a sterol (e.g., cholesterol), and a PEG lipid (e.g., DSPE-PEGSK) can be combined in a desired ratio and dissolved in an organic solvent (e.g., chloroform, methanol, or a combination thereof). A thin film of the lipid materials can be generated by rotary evaporation of the solution in a suitable container (e.g., a glass Florence flask) and dried. Chemical desiccants (e.g., phosphorus pentoxide) or mild heating can be used during the drying process. The lipid film is then hydrated using a suitable buffer (e.g., 10 mM HEPES, pH 7.4) to form of MLVs. The hydration step can be conducted at elevated temperatures (e.g., 65° C.) with or without sonication to aid vesicle formation. The aqueous vesicle suspension can then be extruded through an apparatus equipped with one or more polycarbonate membranes, e.g., a Mini Extruder (Avanti) or LIPEX® Extruder (Transferra). Polycarbonate membranes used in conjunction with such extruders typically range from about 0.03 μm to about 1 μm, and the membrane(s) can be selected to provide liposomes of the desired size. The extrusion can be conducting using a single pass through the membrane(s) or multiple passes through the membrane(s).

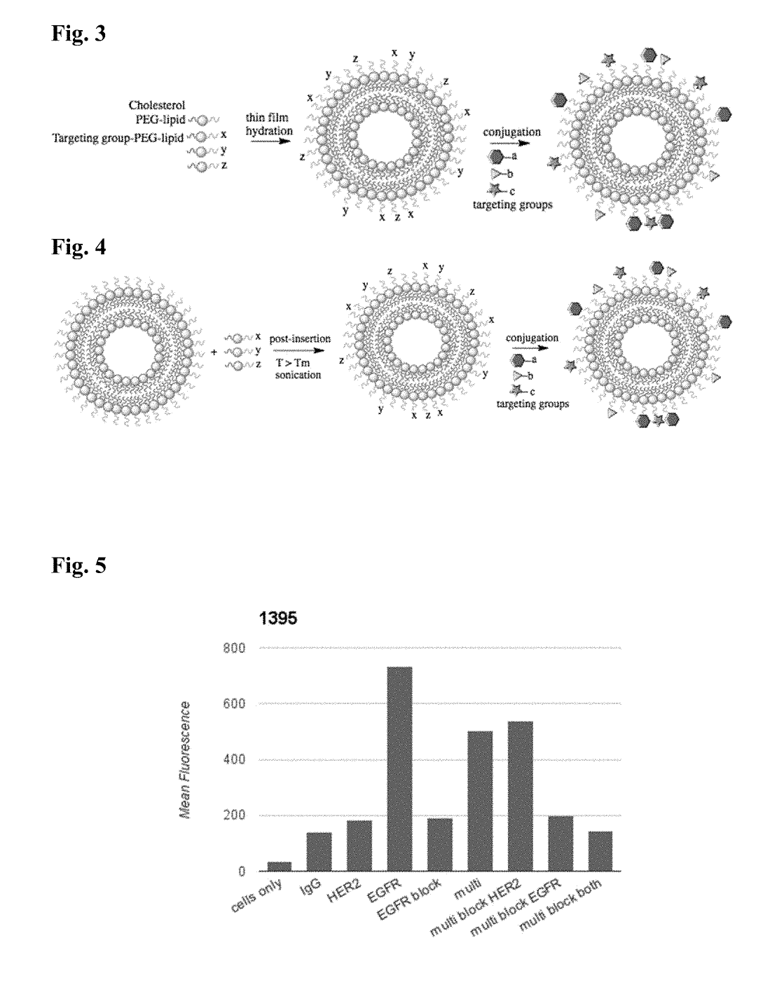

Liposomes containing reactive functional groups can be prepared by combining reactive lipids (e.g., a tetrazine-functionalized phosphatidylethanolamine) with non-reactive lipids (e.g., a sterol, a PEG lipid, a phosphatidyl choline, or a combination thereof) as shown in FIG. 3, prior to the film formation/hydration/extrusion sequence described above.

Alternatively, a non-reactive liposome can be prepared prior to insertion of one or more reactive lipids as shown in FIG. 4. The term “insertion” refers to the embedding of a lipid-functionalized effector element into a liposome bilayer. In general, the lipid-functionalized effector element is transferred from solution to a liposome bilayer due to van der Waals interactions between the hydrophobic portion of the lipid group and the hydrophobic interior of the bilayer. In some embodiments, insertion of the lipid-functionalized effector element is conducted at a temperature above the gel-to-fluid phase transition temperature (Tm) of one or more of the lipid components in the lipid bilayer. The insertion can be conducted, for example, at about 45° C., about 50° C., about 55° C., about 60° C., about 65° C., or at about 70° C. Sonication may also be employed during the insertion process. After post-insertion, the functionalized liposomes can be purified by filtration to remove unincorporated lipids, and effector elements containing the appropriate functional group pair can be conjugated.

By controlling the density of the reactive functional groups on the liposome surface, the amount of each effector element and corresponding conjugates can be tuned. Typically, anywhere between about 100 and 10,000 reactive functional groups will be present on the surface of a given liposome. The click reactions for conjugation can be done in one-pot (i.e., all effector elements added simultaneously), or in series (i.e., each effector element added successively). Following preparation of the functionalized liposomes, effector elements are introduced in one or reactions as described above. As described for the preparation of reactive effector elements, mixtures for conjugating functionalized lipids with effector elements via click reactions can contain additional reagents such as buffers, cosolvents, salts, chelators, reducing agents, and combinations thereof. In general, buffers, cosolvents, salts, chelators, reducing agents, and—in certain instances—detergents/surfactants are employed at concentrations which do not disrupt of the lipid membrane of the liposomes.

Also provided is a method of making a liposome comprising a first effector element and a second effector element via a “post-insertion” process. The method includes combining:
(i) a liposome comprising a lipid membrane with
(ii) a first lipid covalently bonded to the first effector element via a first orthogonal moiety and
(iii) a second lipid covalently bonded to the second effector element via a second orthogonal moiety
under conditions sufficient to insert the first lipid and the second lipid into the lipid membrane;
   wherein the first orthogonal moiety and the second orthogonal moiety have different chemical structures; thereby forming the liposome. In some embodiments, the first effector element and the second effector element are independently selected from the group consisting of a targeting agent, a therapeutic agent, and a diagnostic agent.

In some embodiments, the first orthogonal moiety in the method of making a liposome comprises a dihydropyridazine, a pyridazine, a triazole, a hydrazide, an oxime, a phosphoryl-substituted amide, or a thioether. In some embodiments, the second orthogonal moiety in the method of making a liposome comprises a dihydropyridazine, a pyridazine, a triazole, a hydrazide, an oxime, a phosphoryl-substituted amide, or a thioether, provided that the second orthogonal moiety is different from the first orthogonal moiety. In some embodiments, at least one of the effector elements in the method of making a liposome is a targeting agent. In some embodiments, the targeting agent is selected from an oligopeptide, a polypeptide, an oligonucleotide, and a polynucleotide. In some embodiments, the targeting agent is selected from the group consisting of an antibody and an antibody fragment.

### V. EXAMPLES

### Example 1

### Synthesis of Tetrazine-Functionalized Phospholipid

Methyl-tetrazine-PEG-DSPE was synthesized by conjugation of methyl-tetrazine-NHS with a 5 k DSPE-PEG-amine. Methyl-tetrazine-NHS (1.65 mg), DSPE-PEG-amine (25 mg), and HATU (1.9 mg, Sigma Aldrich) were measured into a glass crimp vial containing a magnetic stir bar. The vial was crimped to close and degassed under dry nitrogen. 250 μL of anhydrous chloroform (Sigma Aldrich) and 3 μL of anhydrous triethylamine were added via syringe. The solution was stirred overnight at room temperature. The resulting solution was purified to remove unreacted methyl-tetrazine and HATU by dialysis with a 3 kDa MWCO membrane. The reaction mixture was dried to remove chloroform, then re-dissolved in a 30% methanol solution and loaded into a 3 kDa MWCO dialysis cassette. The solution was dialyzed overnight in 800 mL of 30% methanol. HPLC was used to determine that no free methyl-tetrazine-NHS or methyl-tetrazine-COOH remained after dialysis. The purified solution was dried under vacuum to provide a bright pink crystalline powder.

### Example 2

### Preparation of Tetrazine-Functionalized Liposomes

Liposomes were prepared using a thin-film evaporation method. 1,2-dipalmitoyl-sn-glycero-3-phosphoethanolamine (DPPE), cholesterol, methoxy-PEG-DSPE (DSPE-mPEG-5K), and DPPE-Cy5.5 were dissolved in a 2:1 chloroform/methanol mix at a molar ratio of 59:36.1:4.8:0.1. The solution was heated to 37° C. to ensure complete dissolution and the solvent was then removed via rotary evaporation at 125 torr. The resulting thin lipid film was further desiccated overnight under vacuum prior to rehydration in phosphate buffered saline (PBS). The rehydrated lipid film was sonicated in a bath sonicator at 69° C. for one hour. While still hot, the lipid solution was extruded through three successive syringe filters (0.45 μm, 0.2 μm, and finally 0.1 μm) to obtain 100-nm liposomes. The liposomes were then washed (by 4-5 rounds of dilution with PBS and concentration via centrifugal filtration with a 100 kDa MWCO membrane or tangential flow filtration (TFF) with a 100 kDa MWCO membrane and 20-30 diavolumes of PBS), to ensure that any unincorporated lipids were removed.

Tetrazine handles were incorporated via post-insertion of a methyl-tetrazine-PEG-DSPE lipid, which was prepared as described above. The liposome sample was divided into four separate samples, to which three separate amounts of tetrazine lipid were added. 0.2, 0.4, 0.6, and 1.0 mg of the tetrazine lipid were used for each liposome sample, respectively. The liposome samples were heated and sonicated at 69° C. for 3 minutes. The tetrazine lipid was dissolved in 200 μL of PBS and then added to the liposome sample. The sample was then heated and sonicated in a sonicator bath at 69° C. for 30 minutes. After sonication, the sample was purified via centrifugal filtration or TFF. The final particles were analyzed for size via dynamic light scattering (DLS), and for size and particle concentration via nanoparticle tracking analysis (NTA, using a Nanosight LM-10).

Tetrazine on the particle surface was quantified via conjugation to a fluorescent dye linked to a trans-cyclooctene (TCO) handle. The dye reacts exclusively with tetrazine and excess is washed off, allowing for quantification of the number of bound dye molecules by measuring fluorescence and calibrating with a standard curve. The conjugation reaction was carried out in 100 μL of PBS containing 5×10¹¹ nanoparticles and Cy3-TCO at 1 mM. A sample of non-tetrazine-containing liposomes was also included as a control for nonspecific adsorption of the Cy3-TCO dye. The reaction was allowed to proceed at room temperature overnight with shaking at 300 rpm. After reacting, unreacted dye was removed via centrifugal filtration using a 100 kDa MWCO filter. NTA was used to determine the nanoparticle concentration after filtration, and the fluorescence of the samples was measured in a fluorescent plate reader along with a standard curve of Cy3-TCO. A linear fit to the standard curve was then used to determine the number of Cy3-TCO molecules bonded to the liposomes, which indicated the number of reactive Tz handles available on the particle surface. The calculated number of Tz handles was then correlated with the initial amount of Tz lipid included in the liposomes, demonstrating a linear relationship and control over the Tz incorporation.

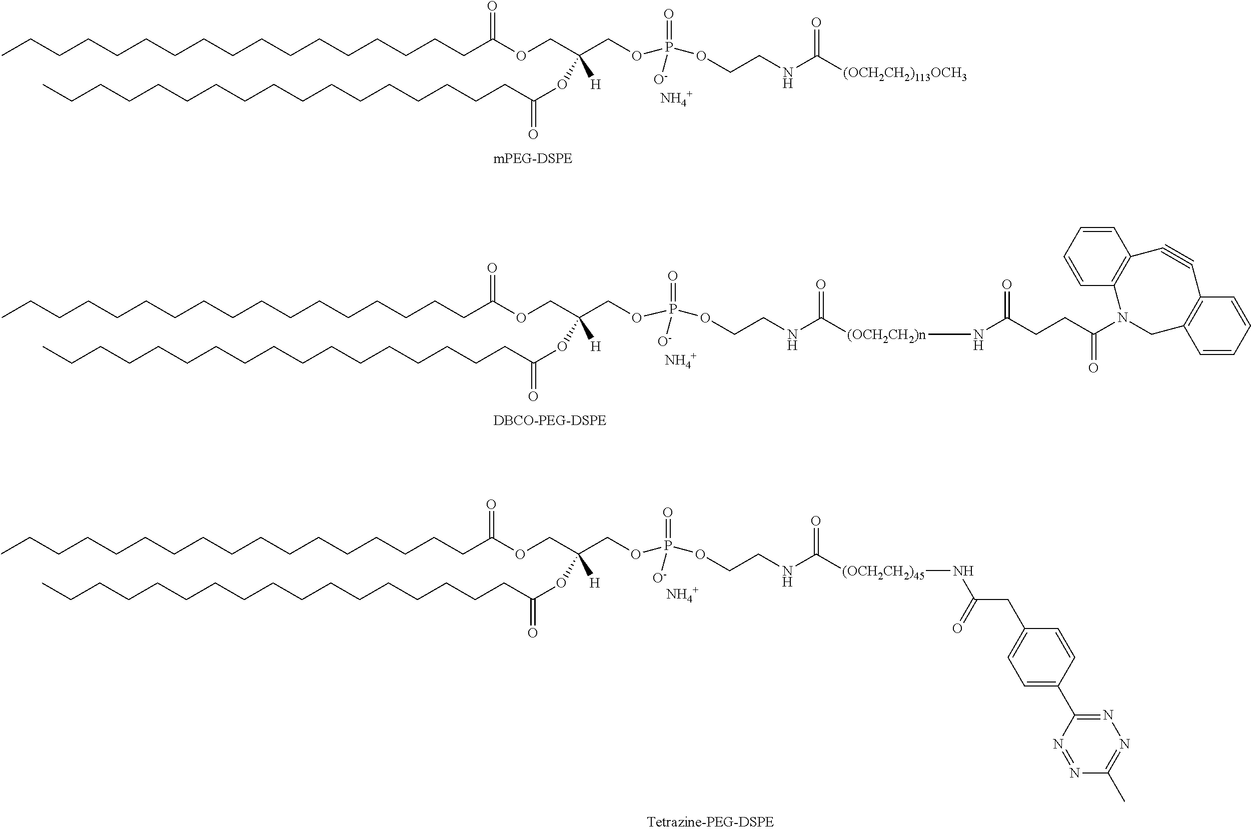

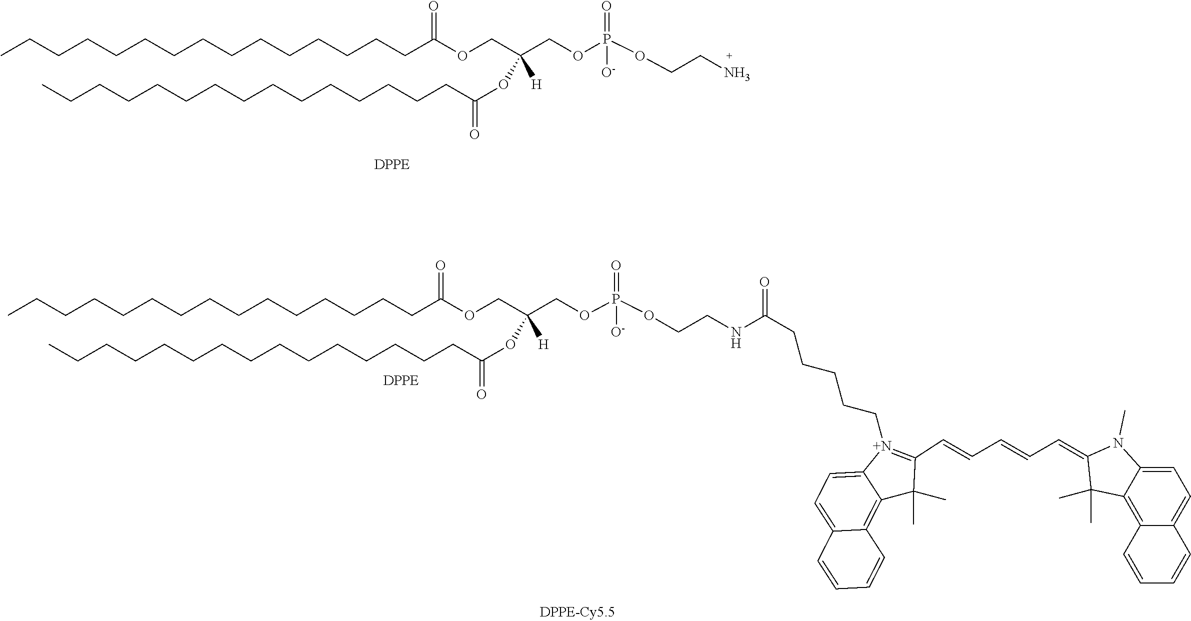 [SMILES: CCCCCCCCCCCCCCCC(=O)OC[C@H](COP(=O)([O-])OCCN)OC(=O)CCCCCCCCCCCCCCC ; CCCCCCCCCCCCCCCC(=O)OC[C@H](COP(=O)([O-])OCCNC(=O)CCCCC[N+]1=C(/C=C/C=C/C=C2/N(C)c3ccc4ccccc4c3C2(C)C)C(C)(C)c2c1ccc1ccccc21)OC(=O)CCCCCCCCCCCCCCC]

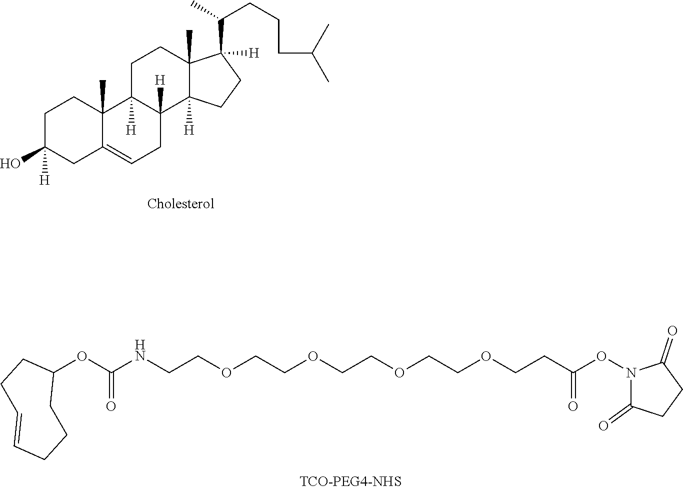

### Example 3

### Preparation of Multifunctional Liposomes

Reactive handles were incorporated into liposomes (prepared as described above) via post-insertion of a methyl-tetrazine-PEG-DSPE lipid, a DBCO-PEG-DSPE-5K lipid, or both. The liposome preparation was divided into three separate samples, to which was added: tetrazine lipid on its own (Tz liposomes); DBCO lipid on its own (DBCO liposomes); or an equal amount of each lipid (multiplexed liposomes). The liposome samples were heated and sonicated at 69° C. for 3 minutes. The tetrazine lipid was dissolved at a concentration of 5 mg/mL in PBS, while the DBCO lipid was dissolved at a concentration of 5 mg/mL in PBS containing 1% DMSO for solubility. 200 μL of Tz lipid was added to one liposome sample, 200 μL of DBCO lipid was added to a second sample, and 100 μL of each lipid was added to a third sample. The samples were then heated and sonicated in a sonicator bath at 69° C. for 30 minutes. After sonication, the samples were purified via either centrifugal filtration or TFF. The final particles were analyzed for size via DLS, and for size and particle concentration via NTA.

Tetrazine and DBCO on the particle surface were quantified by conjugating a dye with the corresponding reactive handle (TCO and azide) and determining the resulting fluorescence of the particles. Reactions were prepared with 5×10¹¹ total nanoparticles in 35 μL final volume. One sample each of the Tz, multiplexed, and PEG liposomes were conjugated in a reaction with 1 mM final concentration of Cy3-TCO (i.e., triethylammonium 2-((E)-3-((E)-1-(6-((3-(((((E)-cyclooct-3-en-1-yl)oxy)carbonyl)amino)propyl)amino)-6-oxohexyl)-3,3-dimethyl-5-sulfonatoindolin-2-ylidene)prop-1-en-1-yl)-1-ethyl-3,3-dimethyl-3H-indol-1-ium-5-sulfonate). One sample each of the DBCO, multiplexed, and PEG liposomes were conjugated in a reaction with 1 mM final concentration of Cy3-azide (i.e., 1-(6-((3-azidopropyl)amino)-6-oxohexyl)-2-((E)-3-((E)-3,3-dimethyl-5-sulfo-1-(3-sulfopropyl)indolin-2-ylidene)prop-1-en-1-yl)-3,3-dimethyl-3H-indol-1-ium-5-sulfonate). Reactions are carried out at room temperature overnight. After the reactions were complete, the samples were purified with 100 kDa MWCO centrifugal filters to remove unconjugated dye. The samples were counted again using NTA and normalized to the same concentration (on a particle number basis). Fluorescence quantification of the samples along with a Cy3 standard curve was used to determine the number of each reactive group on the particles, with the PEG samples accounting for nonspecific adsorption of the dye.

### Example 4

### Cell Recognition by Multiplexed, Antibody-Targeted Liposomes

Commercial anti-HER2 and anti-EGFR antibodies, as well as a non-binding IgG control were decorated with TCO handles in order to enable their conjugation to tetrazine liposomes. The antibodies were modified with both TCO and with a fluorescent marker to enable quantification of the antibodies on the liposomes. TCO was incorporated by reacting 250 μL of 0.4 μg/mL antibody with 100 mM sodium bicarbonate and 13.2 μM TCO-PEG4-NHS (CAS No. 1621096-79-4) for one hour at room temperature. Excess TCO-PEG4-NHS was removed using 30 kDa MWCO centrifugal filters. The number of TCO handles per antibody was then quantified by reacting Cy3-tetrazine (i.e., 1-(6-((4-(1,2,4,5-tetrazin-3-yl)benzyl)amino)-6-oxohexyl)-2-((E)-3-((E)-3,3-dimethyl-5-sulfo-1-(3-sulfopropyl)indolin-2-ylidene)prop-1-en-1-yl)-3,3-dimethyl-3H-indol-1-ium-5-sulfonate) with a small fraction of the prepared TCO-antibody and measuring the absorbance of the dye as compared to the absorbance of the protein at 280 nm. 50 μL of the antibody was prepared at 3 μM, then 3.5 μL of 1 mM Cy3-Tz was added to the solution and allowed to react at room temperature overnight. After reacting, excess dye was removed via centrifugal filtration and the protein's absorbance at 280 and 555 nm was determined. The number of Cy3 moieties bonded to the antibodies was calculated from these absorbance values and the extinction coefficient of the dye. Typically, the procedure resulted in 1.5 TCO handles per antibody.

After conjugating TCO handles to the anti-HER2, anti-EGFR, and control IgG antibodies, the antibodies were labeled with a fluorescent dye. During the wash step after TCO conjugation, the antibodies were concentrated down to 90 μL total volume. To this solution was then added 11 μL of 1 M sodium bicarbonate (final concentration 100 mM) and 7.2 μL of 1 mM fluorescein-5-EX-NHS (i.e., 5-(2-((3-((2,5-dioxopyrrolidin-1-yl)oxy)-3-oxopropyl)thio)-acetamido)-2-(6-hydroxy-3-oxo-3H-xanthen-9-yl)benzoic acid). Excess fluorescein was removed via filtration in a centrifugal filter. This procedure typically resulted in 4-6 fluorescein molecules per antibody.

Tetrazine-decorated particles were produced via post-insertion as described above. These particles were used to produce several different antibody-conjugated samples: anti-HER2 only, anti-EGFR only, anti-HER2 and anti-EGFR, IgG, and a non-conjugated control sample. The “anti-HER2-only” reaction was carried out in 120 μL with 2 μM anti-HER2-TCO and 5×10¹¹ NP/mL in the final solution. The “anti-EGFR-only” reaction was carried out in 120 μL with 1.5 μM anti-EGFR-TCO and 5×10¹¹ NP/mL. The “IgG-only” reaction was carried out in 120 μL with 2 μM IgG-TCO and 5×10¹¹ NP/mL. The “anti-EGFR-TCO+anti-HER2-TCO” reaction was carried out in 120 μL with 0.75 μM anti-EGFR, 1 μM anti-HER2, and 5×10¹¹ NP/mL. All reactions took place overnight at room temperature. After the reactions took place, unreacted antibody was removed by washing the particles in 300 kDa MWCO centrifugal filters. After conjugation and purification, the liposomes were analyzed for size and concentration via NTA. The number of antibodies per liposome was quantified by determining the fluorescein fluorescence of the particles and correlating with standard curves prepared from each antibody. An average number of about 52 antibodies per liposome was determined.

TABLE 1
Incorporation of multiple orthogonal chemistries on a liposome.

| Sample | Tz/NP | DBCO/NP |
|---|---|---|
| Tz-PEG-DSPE only | 1958 | 0 |
| DBCO-PEG-DSPE only | 0 | 1239 |
| Tz-PEG-DSPE and DBCO-PEG- | 1487 | 1106 |
| DSPE | | |

Cell-binding experiments were carried out with four different human cell lines: HCC1395, which overexpresses EGFR but does not express HER2; BT-454, which overexpresses HER2 but does not express EGFR; ZR-75-1, which expresses both HER2 and EGFR; and OVSAHO, which does not express either receptor. All cell binding was carried out with cells in suspension at 10⁶ cells/mL and 10¹¹ NP/mL. “Blocking” samples were pre-incubated for 30 minutes at 37° C. with an additional 2.5 μL of 0.5 μg/mL anti-EGFR or anti-HER2, unconjugated and unlabeled with dye. Liposomes were incubated with cells at 37° C. for 1.5 hours, then the cells were gently pelleted and washed with PBS to remove unbound liposomes. Cells were resuspended in 200 μL of PBS and run on an Attune flow cytometer. Shifts in fluorescence versus cells only were observed in the fluorescein (antibody) and Cy5.5 (particle) channels. Flow cytometry data was analyzed in FlowJo to determine the geometric mean in each channel, as a quantitative comparison of the degree of binding by each sample to each cell type.

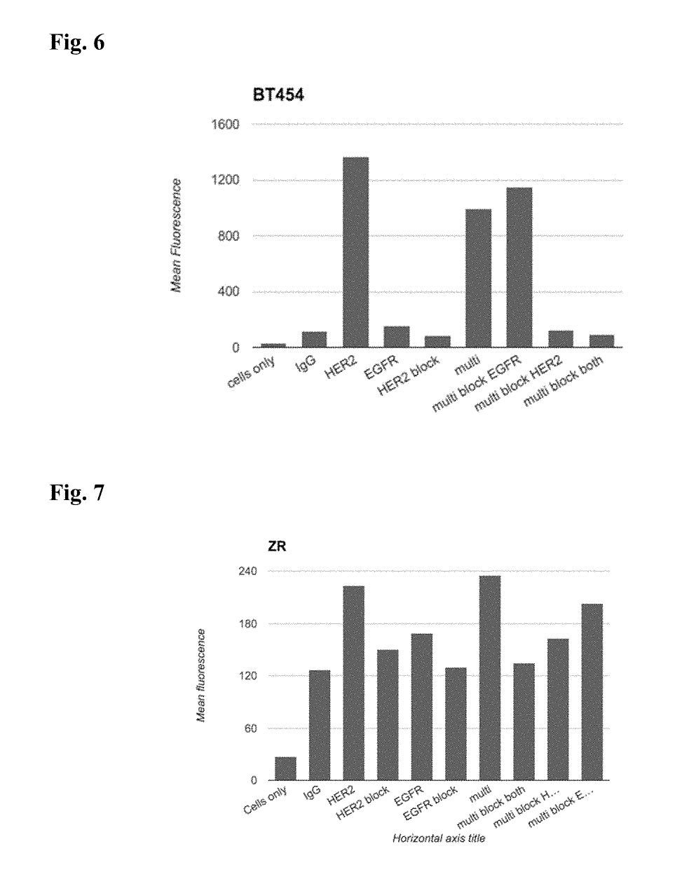

The dual-targeting liposomes showed significant binding to two different cell lines, HCC1395 (overexpresses EGFR) and BT454 (overexpresses HER2), as well as increased binding to ZR751, a cell line with moderate expression of both receptors. These results are shown in FIGS. 5, 6, and 7, with plots of the mean fluorescence for cells incubated with IgG (non-binding), anti-HER2 only, anti-EGFR, and both anti-HER2 and anti-EGFR liposomes (indicated as IgG, HER2, EGFR, or multi). Blocking of cellular receptors with free antibody (indicated by “liposome name block antibody”, such as multi block EGFR) was performed to demonstrate that the fluorescence increase was indeed the result of the liposome binding to the desired receptor.

Although the foregoing has been described in some detail by way of illustration and example for purposes of clarity and understanding, one of skill in the art will appreciate that certain changes and modifications can be practiced within the scope of the appended claims. In addition, each reference provided herein is incorporated by reference in its entirety to the same extent as if each reference was individually incorporated by reference.

1. A particle comprising a first effector element covalently bonded to a nanoparticle via a first orthogonal moiety and a second effector element covalently bonded to the nanoparticle via a second orthogonal moiety, wherein the first orthogonal moiety and the second orthogonal moiety have different chemical structures.

2. The particle of claim 1, wherein the first effector element and the second effector element are independently selected from the group consisting of a targeting agent, a therapeutic agent, and a diagnostic agent.

3. The particle of claim 1, wherein the first orthogonal moiety comprises a dihydropyridazine, a pyridazine, a triazole, a hydrazide, an oxime, a phosphoryl-substituted amide, or a thioether, and
wherein the second orthogonal moiety comprises a dihydropyridazine, a pyridazine, a triazole, a hydrazide, an oxime, a phosphoryl-substituted amide, or a thioether, provided that the second orthogonal moiety is different from the first orthogonal moiety.

4. The particle of claim 1, further comprising a first linking moiety between the first orthogonal moiety and the nanoparticle, and optionally further comprising a second linking moiety between the second orthogonal moiety and the nanoparticle.

5. The particle of claim 4, wherein the first linking moiety comprises an oligo(ethylene glycol) or a poly(ethylene glycol), and wherein the second linking moiety, when present, comprises an oligo(ethylene glycol) or a poly(ethylene glycol).

6-9. (canceled)

10. The particle of claim 1, wherein at least one of the first effector element and the second effector element is a targeting agent.

11. The particle of claim 10, wherein the targeting agent is selected from the group consisting of an oligopeptide, a polypeptide, an oligonucleotide, and a polynucleotide.

12. The particle of claim 10, wherein the targeting agent is selected from the group consisting of an antibody and an antibody fragment.

13. The particle of claim 1, wherein the nanoparticle is a liposome comprising a lipid membrane, wherein the first orthogonal moiety further comprises a first lipid group and the second orthogonal moiety further comprises a second lipid group, and wherein the first lipid group and the second lipid group are embedded in the lipid membrane.

14-17. (canceled)

18. The particle of claim 1, further comprising a therapeutic agent, diagnostic agent, or a combination thereof.

19. A population of particles according to claim 1.

20. The population of particles according to claim 19, wherein the average diameter of the particles is 100 nanometers or less.

21. The population of particles according to claim 19, wherein the polydispersity index of the population is 0.20 or less.

22. A method of making a particle comprising a first effector element and a second effector element, the method comprising:
(i) providing a nanoparticle having a first reactive functional group and a second reactive functional group; and
(ii) combining the nanoparticle with a first reactive effector element and a second reactive effector element under conditions sufficient to form:
(a) a first orthogonal moiety covalent bonding the first reactive functional group to the first reactive recognition element and
(b) a second orthogonal moiety covalently bonding the second reactive functional group to the second reactive effector element;
wherein the first orthogonal moiety and the second orthogonal moiety have different chemical structures;
thereby forming the particle.

23. The method of claim 22, wherein the first effector element and the second effector element are independently selected from the group consisting of a targeting agent, a therapeutic agent, and a diagnostic agent.

24. The method of claim 22, wherein the first reactive functional group is selected from the group consisting of a cycloalkyne, a linear alkyne, a cycloalkene, a tetrazine, an aminooxy compound, a hydrazide, a ketone, an azide, a phosphine, a thiol, and a maleimide, and
wherein the second reactive functional group is selected from the group consisting of a cycloalkyne, a linear alkyne, a cycloalkene, a tetrazine, an aminooxy compound, a hydrazide, a ketone, an azide, a phosphine, a thiol, and a maleimide, provided that the second reactive functional group is different from the first reactive functional group.

25-32. (canceled)

33. The method of claim 22, wherein the nanoparticle is a liposome comprising a lipid membrane, wherein the first orthogonal comprises a first lipid group and the second orthogonal moiety, when present, comprises a second lipid group, and wherein the first lipid group and the second lipid group are embedded in the lipid membrane.

34-35. (canceled)

36. A method of making a liposome comprising a first effector element and a second effector element, the method comprising combining:
(i) a liposome comprising a lipid membrane with
(ii) a first lipid covalently bonded to the first effector element via a first orthogonal moiety and
(iii) a second lipid covalently bonded to the second effector element via a second orthogonal moiety
under conditions sufficient to insert the first lipid and the second lipid into the lipid membrane,
wherein the first orthogonal moiety and the second orthogonal moiety have different chemical structures,
thereby forming the liposome.

37. The method of claim 36, wherein the first effector element and the second effector element are independently selected from the group consisting of a targeting agent, a therapeutic agent, and a diagnostic agent.

38. The method of claim 36, wherein the first orthogonal moiety comprises a dihydropyridazine, a pyridazine, a triazole, a hydrazide, an oxime, a phosphoryl-substituted amide, or a thioether, and wherein the second orthogonal moiety comprises a dihydropyridazine, a pyridazine, a triazole, a hydrazide, an oxime, a phosphoryl-substituted amide, or a thioether, provided that the second orthogonal moiety is different from the first orthogonal moiety.

39-42. (canceled)
